(19) United States
(12) Patent Application Publication
(10) Pub. No.: US 2022/0339021 A1
(43) Pub. Date: Oct. 27, 2022

(54) PERCUTANEOUS TRANSABDOMINAL PORT FOR HOLLOW VISCERA

(71) Applicant: SIMOVATIONS INC., Denver, CO (US)

(72) Inventor: Sarah HECHT, Denver, CO (US)

(21) Appl. No.: 17/762,004

(22) PCT Filed: Sep. 25, 2020
(86) PCT No.: PCT/US2020/052651

§ 371 Date: Mar. 18, 2022

Related U.S. Application Data
(60) Provisional application No. 62/905,978, filed on Sep. 25, 2019.

(51) Int. Cl. A61F 5/445; A61F 5/44

(52) U.S. Cl. CPC A61F 5/445; A61F 5/4405; A61F 2005/4455

(57) ABSTRACT

Disclosed is a percutaneous transabdominal port that provides access to a hollow visceral organ. The percutaneous transabdominal port is hollow and has a channel connecting the external surface of the abdomen to the inside of an organ. A catheter or other instrument may pass through this channel into the organ. The percutaneous transabdominal port has a hollow tube which spans an abdominal wall tract, an internal retainer to prevent unintended removal, and an external retainer to prevent withdrawal into the body. If the internal retainer does not rely on inflation, the hollow tube may be cut to length. An optional seal prevents fluid leakage while permitting passage of catheters or instruments. An optional closure cap is described. A method of inserting, utilizing, and removing the percutaneous transabdominal port is described.

### CLAIM TO PRIORITY

The present document claims priority to U.S. Provisional Patent Application 62/905,978 filed 25 Sep. 2019, the contents of which is hereby incorporated by reference in its entirety.

### FIELD

The present document relates to transabdominal ports, and more particularly to a catheterizable port for bladder, colonic, or gastric access and drainage.

### BACKGROUND

Many people suffer from various bladder, colonic, or gastric conditions which require long-term access to internal organs of the body. This access is necessary for instillation of nutrients, fluids, or other substances; organ drainage or decompression; and/or endoscopic procedures. One relatively common situation is the need for access to the urinary bladder to drain urine. Urinary conditions may prevent or at least inhibit conventional urination. If a person has a neurogenic bladder, the bladder may be unable to contract and is thus unable to discharge urine. Others may suffer from a urinary tract blockage or may have other challenges that impede the ability to urinate. When urine cannot pass naturally, a person must use catheters to drain urine from the bladder, and/or undergo surgical creation of a passage from the urinary system to the skin through which urine can exit. These currently available solutions have significant shortcomings and drawbacks.

Conventional urinary catheters may access the bladder in two ways. A catheter may be passed through the urethra into the bladder. Alternatively, a catheter may be inserted into the bladder through a surgically created connection, called a suprapubic cystostomy, between the bladder and the external abdomen. Catheters inserted through the urethra may be either constantly indwelling or passed intermittently through the urethra into the bladder multiple times per day. Catheters inserted through a suprapubic cystostomy are constantly indwelling; these are referred to as suprapubic catheters.

There are myriad shortcomings to these conventional bladder drainage solutions. Some complications associated with standard urethral catheters include:
Catheter blockage (from biofilms, encrustation, mucus, debris, blood clots, tube kinking)
Urinary infection
Traumatic insertion
Urethral stricture
Pressure wounds such as traumatic hypospadias (fileting open of the penis or female urethra due to traction on the catheter)
Issues with tubing and bags associated with the catheter (cosmesis, discretion, kinking, leaks, cumbersome nature)
Testicular, epididymal, and prostate infection/inflammation induced by catheter use
Balloon complications such as bladder irritation, failure to deflate, rupture with retained balloon fragments, bladder cancer
Pain and discomfort
Preclusion of sexual intercourse due to physical interference of the catheter with intercourse
Lack of independence because many patients need someone to catheterize for them due to difficulty accessing urethra or problems with dexterity
Lack of privacy and discretion because most catheters are inserted through genitalia
Poor urine drainage

Suprapubic catheters have the advantage of avoiding urethral and genital complications but are subject to similar non-genital non-urethral complications. Additionally, they are bulky and protrude from the skin, and are thus indiscreet. Further, catheters are mobile within the suprapubic cystostomy tract, and movement leads to widening of the tract, which in turn leads to urine leakage and skin complications such as granulomas, infections, and skin breakdown.

Other existing percutaneous tubes, such as cecostomy tubes or gastrostomy tubes, suffer from tract complications similar to those encountered with suprapubic catheters, including complications arising from motion within the tract. To avoid motion of a device relative to the tract, approximate sizing is inadequate; a device must be securely anchored either via compression between precisely placed internal and external bolsters, or secured with an accessory method such as tape. Further, such tubes are primarily designed for fluid instillation and drain poorly. In addition to precluding adequate drainage, such a narrow channel precludes efficient organ irrigation, which is sometimes necessary for removal of debris, mucus, and blood clots.

### SUMMARY

A percutaneous transabdominal port adapted for access to abdominal hollow organs is disclosed. Such a port comprises a hollow tube with an external diameter small enough to fit through a tract within an abdominal wall, and an internal retainer connected with the internal end of the hollow tube. The internal retainer has a first configuration with a first external diameter small enough to fit through the abdominal wall tract, and a second configuration with a second external diameter greater than the first external diameter. When the percutaneous transabdominal port is installed in a patient, the hollow tube is within the abdominal wall tract, and the internal retainer is within a hollow organ in its second, wider configuration so as to prevent movement of the port out of the organ. The hollow tube has an open external end and an open internal end such that instrumentation may access the organ by passing through the hollow tube and into the organ.

The internal retainer may be inflatable such as a balloon, or it may be elastomeric and resiliently deformable. The shape of the internal retainer may be converted to a first, narrow configuration for insertion and removal, and converted to a second, wide configuration when positioned in an organ. An inflatable internal retainer may be deflated to a first, narrow configuration and inflated to a second, wide configuration. A deformable internal retainer may be deformed to a first, narrow configuration when mechanical force is applied to an interior surface of the internal retainer. A deformable internal retainer may also be deformed to a narrow configuration when a mechanical force is applied to an outer surface of the retainer. In both cases, removal of the mechanical force allows the retainer to assume a second, wider configuration.

The percutaneous transabdominal port may further include an external retainer which couples to the external end of the hollow tube. Such an external retainer has a diameter larger than the diameter of both the hollow tube and the abdominal wall tract so as to prevent retraction of the percutaneous transabdominal port into the abdominal wall tract. The percutaneous transabdominal port may further comprise a closure cap to seal the hollow tube external to the abdominal wall.

The percutaneous transabdominal port may further include at least one seal. Such a seal prevents fluid leakage from the organ out of the port. The seal also accommodates an instrument passing through the percutaneous transabdominal port, forming an elastomeric seal to the instrument.

Methods for inserting, utilizing, and removing the percutaneous transabdominal port are additionally disclosed. The hollow tube and the internal retainer of the percutaneous transabdominal port may be inserted through the abdominal wall tract when the internal retainer is in the first, narrow configuration. The port with the internal retainer in its first, narrow configuration is passed through the abdominal wall tract until the internal retainer is contained within the organ. The internal retainer is then reconfigured to its second, wider configuration such that the internal retainer does not move out of the organ (and such that the port is fixed in place). An external retainer may be integrally formed with the external end of the hollow tube. Alternatively, the external retainer may be secondarily coupled to the external end of the hollow tube. In the latter example, the hollow tube may be shortened such that the percutaneous transabdominal port may be custom-sized to a patient's abdominal wall thickness.

To remove the percutaneous transabdominal port, the internal retainer is converted to its first, narrow configuration. The percutaneous transabdominal port may then be withdrawn from the organ through the abdominal wall tract and out of the body.

An instrument may be passed through the percutaneous transabdominal port. An optional closure cap may be opened. An instrument may then be passed through the channel of the hollow tube and into the organ. If the percutaneous transabdominal port contains a seal, the instrument passes through this seal. The instrument may then be withdrawn from the percutaneous transabdominal port. An optional closure cap may then be closed when the port is not is use.

### BRIEF DESCRIPTION OF THE DRAWINGS

FIG. 1 shows a profile view of the percutaneous transabdominal port inserted into a patient, in accord with an embodiment.

FIG. 2A shows a cross-sectional frontal view of an embodiment of the hollow tube and internal retainer of the percutaneous transabdominal port, which are integrally formed, including a seal formed within the retainer tip.

FIG. 2B shows a cross-sectional perspective view of an embodiment of the hollow tube and internal retainer of the percutaneous transabdominal port, which are integrally formed, including a seal which is formed with the retainer tip.

FIG. 3A shows a cross-sectional frontal view of a duckbill valve.

FIG. 3B shows a cross-sectional perspective view of a duckbill valve.

FIG. 3C shows a top view of a duckbill valve.

FIG. 4 shows a cross-sectional frontal view of an instrument (catheter) passing through a duckbill valve.

FIG. 5 shows an example of a cross-slit valve.

FIG. 6 shows an example of a diaphragm valve.

FIG. 7 shows an example of a flapper valve.

FIG. 8 shows an embodiment of a percutaneous transabdominal port with an inflatable internal retainer in a deflated, narrow configuration.

FIG. 9 shows an embodiment of a percutaneous transabdominal port with an inflatable internal retainer in an inflated, wide configuration.

FIG. 10 shows a cross-sectional frontal view of a percutaneous transabdominal port with an inflatable internal retainer in an inflated, wide configuration, including a seal which is formed with the hollow tube.

FIG. 11 shows a frontal view of a deformable internal retainer in an expanded configuration, in an embodiment.

FIG. 12 shows a frontal view of a deformable internal retainer in the narrowed configuration, in an embodiment.

FIG. 13 shows a cross-sectional frontal view of a deformable internal retainer, in an embodiment.

FIG. 14A shows a cross-sectional frontal view of an embodiment of a deformable internal retainer with internal guiding sidewalls extending down from the hollow tube and up from the retainer tip into a hollow space of the internal retainer, in an embodiment.

FIG. 14B shows a cross-sectional perspective view of an embodiment of a deformable internal retainer with internal guiding sidewalls extending down from the hollow tube and up from the retainer tip into a hollow space of the internal retainer, in an embodiment.

FIG. 15 shows a cross-sectional frontal view of an embodiment of a deformable internal retainer, where the channel diameter is larger than the opening in the tip of the internal retainer and where the internal retainer tip is reinforced with a flat shelf surrounding the opening.

FIG. 16A shows a cross-sectional frontal view of an embodiment of a deformable internal retainer, where the channel diameter is larger than the opening in the tip of the internal retainer, and where the internal retainer tip has an integrated duckbill valve with a flange.

FIG. 16B shows a cross-sectional perspective view of an embodiment of a deformable internal retainer, where the channel diameter is larger than the opening in the tip of the internal retainer, and where the internal retainer tip has an integrated duckbill valve with a flange.

FIG. 17A shows a perspective view of an embodiment of a deformable internal retainer with a closed tip and apertures in the retainer sidewall.

FIG. 17B shows a cross-sectional frontal view of an embodiment of a deformable internal retainer with a closed tip and apertures in the retainer sidewall.

FIG. 18 details a method to deform an internal retainer to a narrow configuration using straightening rods, in an embodiment.

FIG. 19 shows fingers engaging optional finger tabs as the thumb engages a straightening rod push platform, in an embodiment.

FIG. 20 shows fingers engaging optional finger tabs as a thumb pushes a straightening rod push platform to narrow a deformable internal retainer, in an embodiment.

FIG. 21 shows one embodiment of a straightening rod, intended for use with a deformable internal retainer with a tip opening smaller than the channel diameter.

FIG. 22 shows a cross-sectional frontal view of a straightening rod engaging the tip of a deformable internal retainer without passing through the tip opening, in an embodiment.

FIG. 23 shows a cross-sectional frontal view of a deformable internal retainer deforming in response to a straightening rod applying axial pressure to the retainer tip, in an embodiment.

FIG. 24 shows one embodiment of a straightening rod with a hollow tip, intended for use with a deformable internal retainer with sidewalls extending up from the internal retainer tip.

FIG. 25 shows a cross-sectional frontal view of a straightening rod with a hollow tip engaging internal sidewalls at the tip of a deformable internal retainer, in an embodiment.

FIG. 26 shows a cross-sectional frontal view of a deformable internal retainer deforming in response to a straightening rod applying axial pressure to the retainer tip, in an embodiment.

FIG. 27 shows one embodiment of a straightening rod, intended for use with a deformable internal retainer with a closed tip.

FIG. 28 shows a straightening rod engaging the closed tip of a deformable internal retainer, in an embodiment.

FIG. 29 shows a deformable internal retainer deforming in response to a straightening rod applying axial pressure to the closed internal retainer tip, in an embodiment.

FIG. 30 shows a cross-sectional frontal view of a portion of a trocar surrounding a deformable internal retainer, constraining the internal retainer in a narrow configuration.

FIG. 31 shows a profile view of a soluble capsule surrounding a deformable internal retainer, constraining the internal retainer in a narrow configuration.

FIG. 32 shows a profile view of one embodiment of an external retainer in which the collar extends upward from the flange.

FIG. 33 shows a profile view of one embodiment of an external retainer in which the collar extends downward from the flange.

FIG. 34 shows a profile view of one embodiment of an external retainer in which the collar extends both upward and downward from the flange.

FIG. 35 shows an embodiment of an external retainer flange which is continuous and disc-like.

FIG. 36 shows an embodiment of an external retainer flange which is notched and petal-like.

FIG. 37 shows an embodiment of an external retainer flange which is discontinuous and wing-like.

FIG. 38 shows a profile view of one embodiment of an external retainer in which the flange curvature is convex relative to the abdomen.

FIG. 39 shows a profile view of one embodiment of an external retainer in which the flange curvature is concave relative to the abdomen.

FIG. 40 shows a sectional perspective view of circumferential angled ridges protruding from the inside of an external retainer collar, in an embodiment.

FIG. 41 shows a sectional perspective view of circumferential angled grooves in the outer wall of the hollow tube, in an embodiment.

FIG. 42 shows a profile view of the angled ridges of an external retainer engaging the angled grooves of a hollow tube, where grooves and ridges mate to create a one-way ratcheting system to secure the external retainer to the hollow tube, in an embodiment.

FIG. 43 shows a top view of an external retainer collar with locking pushpins in an open configuration, in an embodiment.

FIG. 44 shows a top view of an external retainer collar with locking pushpins in a locked configuration, in an embodiment.

FIG. 45 shows a profile view of an external retainer collar with locking pushpins in an open configuration surrounding a hollow tube, which has depressions to receive the pushpins, in an embodiment.

FIG. 46 shows a profile view of an external retainer collar surrounding a hollow tube, the pushpins having engaged the depressions within the hollow tube wall to secure the external retainer, in an embodiment.

FIG. 47 shows a profile view of an external retainer collar surrounding a hollow tube, where snap fit tabs on the external retainer are deflected away from depressions within the hollow tube wall, in an embodiment.

FIG. 48 shows a profile view of an external retainer collar surrounding a hollow tube, where snap fit tabs on the external retainer are engaged in depressions within the hollow tube wall to secure the external retainer, in an embodiment.

FIG. 49 shows a top view of an embodiment of an external retainer collar with internal protrusions, configured for a twist-lock.

FIG. 50 shows a top view of an embodiment of a hollow tube with longitudinal slots, configured for a twist-lock.

FIG. 51 shows a perspective view of a hollow tube with longitudinal slots and perpendicular semi-circumferential grooves, configured for a twist-lock, in an embodiment.

FIG. 52 shows a top view of external retainer protrusions engaged with slots within the hollow tube wall; once positioned, the external retainer is rotated and the protrusions engage the semi-circumferential grooves to secure the retainer, in accord with an embodiment.

FIG. 53 shows a profile view of a protrusion from an external retainer collar engaged in a locked position within a semi-circumferential groove, in an embodiment.

FIG. 54 shows an embodiment of a flip-plug closure in the open position.

FIG. 55 shows a profile view of the plug inserting into the channel in the closed position, in an embodiment.

FIG. 56 shows an embodiment of a flip-cap closure in the open position.

FIG. 57 shows a profile view of the cap engaging the hollow tube walls in the closed position, in an embodiment.

FIG. 58 details a method to insert a percutaneous transabdominal port in which the hollow tube, internal retainer, external retainer, and closure cap are integrally formed, in accord with an embodiment.

FIG. 59 shows a percutaneous transabdominal port in which the hollow tube, internal retainer, external retainer, and closure cap are integrally formed prior to insertion, in an embodiment.

FIG. 60 shows a percutaneous transabdominal port in which the hollow tube, internal retainer, external retainer, and closure cap are integrally formed, inserted through an abdominal wall tract into an organ with its internal retainer in the narrow configuration, in an embodiment.

FIG. 61 shows a percutaneous transabdominal port in which the hollow tube, internal retainer, external retainer, and closure cap are integrally formed, inserted through an abdominal wall tract into an organ with its internal retainer in the wide configuration, in an embodiment.

FIG. 62 shows an implanted percutaneous transabdominal port with an instrument (e.g. catheter) passing through the port into an organ, in an embodiment.

FIG. 63 shows an example of an embodiment of a percutaneous transabdominal port in which the hollow tube and internal retainer are integrally formed; also shown are finger tabs, integrally formed with the external end of the hollow tube.

FIG. 64 shows an example of an external retainer which is integrally formed with a closure cap, in an embodiment.

FIG. 65 shows an example of an embodiment of a percutaneous transabdominal port in which the hollow tube and internal retainer with finger tabs are integrally formed; a closure cap and external retainer are separately integrally formed and positioned around the hollow tube.

FIG. 66 details a method to insert a percutaneous transabdominal port in which the hollow tube and internal retainer are integrally formed, and where the closure cap and external retainer are separately integrally formed, allowing for shortening of the hollow tube to custom size the device, in accord with an embodiment.

FIG. 67 shows a cross-sectional frontal view of an embodiment of a percutaneous transabdominal port with an integrally formed hollow tube and internal retainer and separately integrally formed closure cap and external retainer.

FIG. 68 shows a cross-sectional frontal view of a straightening rod engaging the tip of an external retainer of a percutaneous transabdominal port, in an embodiment.

FIG. 69 shows a cross-sectional frontal view of a straightening rod inserted into a percutaneous transabdominal port with an integrally formed hollow tube and internal retainer and separately integrally formed closure cap and external retainer, the internal retainer being in the narrow configuration, in accord with an embodiment.

FIG. 70 shows a cross-sectional frontal view of an embodiment of the percutaneous transabdominal port with an integrally formed hollow tube and internal retainer and separately integrally formed closure cap and external retainer being inserted through the abdominal wall tract into an organ.

FIG. 71 shows a cross-sectional frontal view of an embodiment of a percutaneous transabdominal port with an integrally formed hollow tube and internal retainer and separately integrally formed closure cap and external retainer which has been inserted into the organ.

FIG. 72 shows a cross-sectional frontal view of an embodiment of a percutaneous transabdominal port with an internal retainer appropriately positioned within the organ against the internal abdominal wall.

FIG. 73 shows a cross-sectional frontal view of a percutaneous transabdominal port with a closure cap integrally formed with an external retainer positioned against the external abdominal wall surface, in an embodiment.

FIG. 74 shows a cross-sectional frontal view of an embodiment of a percutaneous transabdominal port appropriately positioned within the abdominal wall tract; excess hollow tube has been removed.

FIG. 75 shows a frontal view of an embodiment of a percutaneous transabdominal port appropriately positioned within the abdominal wall tract; excess hollow tube has been removed, and the cap is in the closed position.

FIG. 76 shows a cross-sectional frontal view of a previously implanted percutaneous transabdominal port with a straightening rod inserted to convert the internal retainer to a narrow configuration for removal, in accord with an embodiment.

FIG. 77 shows a cross-sectional frontal view of a previously implanted percutaneous transabdominal port being withdrawn from the abdominal tract, in accord with an embodiment.

### DETAILED DESCRIPTION

The percutaneous transabdominal port disclosed herein is a medical device connecting a hollow internal organ, such as the urinary bladder, bowel or stomach, to the external surface of the abdomen, which includes the suprapubic region. The percutaneous transabdominal port is intended to be catheterizable, such that a medical catheter or another instrument, such as an endoscope, may be passed through the port into the organ.

The percutaneous transabdominal port of one embodiment has several parts. The first part is a hollow tube with a channel that traverses a tract between the organ and the external abdominal wall. The second part is an internal retainer attached to the internal end of the hollow tube and configured to prevent movement of the percutaneous transabdominal port out of the organ. The third part is an external retainer attached to the external end of the hollow tube and configured to prevent movement of the percutaneous transabdominal port into the body. The fourth part is an optional closure cap to seal the percutaneous transabdominal port when not in use. The percutaneous transabdominal port may further include a seal to prevent fluid leakage.

Also disclosed herein is a method for inserting the percutaneous transabdominal port into a patient and a method for removing the percutaneous transabdominal port from a patient.

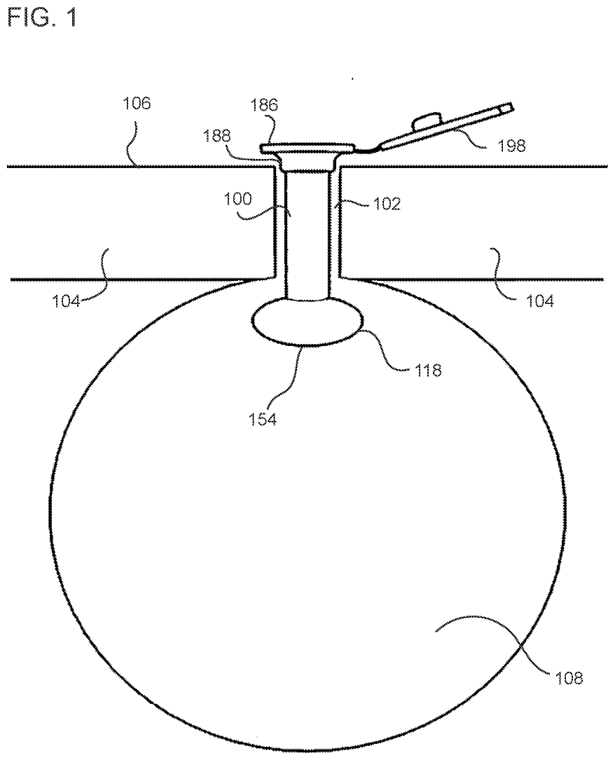

Referring to the drawings, an embodiment of the percutaneous transabdominal port implanted in a patient is depicted in FIG. 1. The hollow tube 100 of the percutaneous transabdominal port sits within a tract 102 in the patient's abdominal wall 104 which connects the exterior surface of the abdomen 106 with the interior space of the hollow organ 108. The hollow tube 100 length must equal or exceed the length of the abdominal wall tract 102. Therefore, the hollow tube 100 has a length such as between 0.5 cm and 50 cm, depending on the thickness of a patient's abdominal wall.

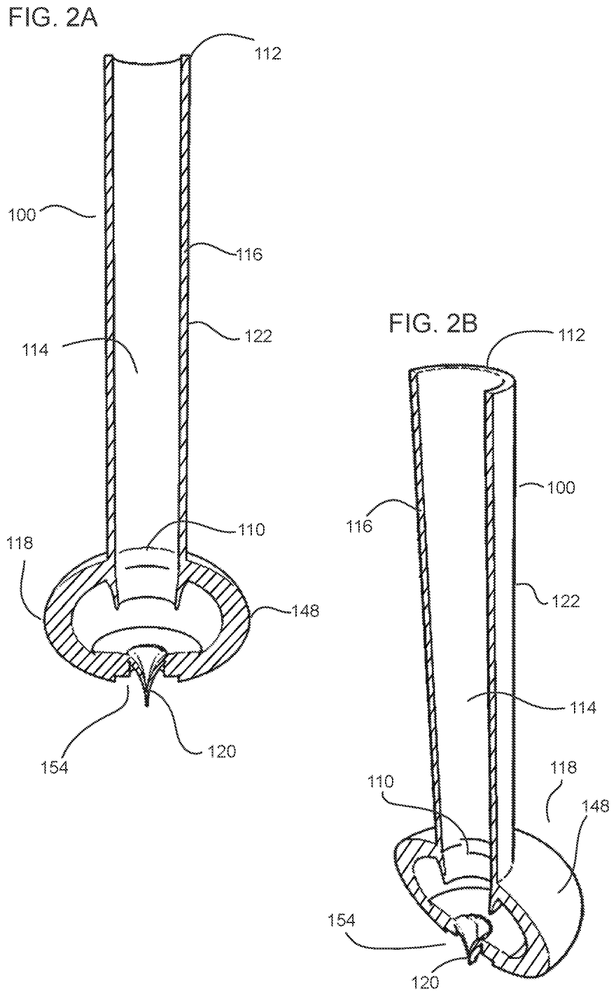

Referring now to FIGS. 2A-B, the hollow tube 100 has an open internal end 110 and an open external end 112. In some embodiments, the channel 114 within the hollow tube has a diameter sized to accommodate passage of a catheter or other instrument. Standard medical catheters range in diameter from 3.5 French (1.2 millimeter) feeding tubes to 36 French (12 millimeter) urinary catheters. Instruments such as rigid nephroscopes, colonoscopes, or anoscopes may have diameters up to 54 French (18 millimeters). The channel 114 may thus have a diameter such as between 4 French (1.3 millimeters) and 60 French (20 millimeters) to permit passage of such catheters and instruments. Some embodiments may provide a larger channel to accommodate passage of a larger instrument.

The thickness of the hollow tube wall 116 will vary based on its constitutive material. It should be thick enough to provide adequate rigidity; a hollow tube of adequate rigidity resists the compressive forces of the abdominal wall tract without deforming the channel 114. In general, the hollow tube wall 116 thickness will range from 1 millimeter-10 millimeters. The hollow tube wall 116 thickness may be uniform or nonuniform along the length of the hollow tube 100. In one embodiment, variation in wall thickness may provide variation in rigidity to counter varying compressive forces along different portions of the tract. One such embodiment has increased wall thickness of the hollow tube 100 toward its external end 112 to counteract the tendency for a tract to stenose at the skin surface. In some embodiments, the hollow tube 100 is tapered. In one such embodiment, the outer diameter of the external end 112 of the hollow tube 100 is larger than the outer diameter of the internal end 110 of the hollow tube 100. Such an embodiment with a tapered internal end 110 may facilitate smooth insertion of the percutaneous transabdominal port through an abdominal wall tract 102.

In some embodiments, the hollow tube 100 is constructed from biocompatible elastomeric materials via extrusion or injection molding. Exemplary materials include silicone, polyurethane, polyvinylchloride, and latex. The hollow tube 100 may also be entirely constructed of, or have elements constructed from, biocompatible plastic material. Exemplary materials include polyetheretherketone, polypropylene, and polytetrafluoroethylene. In an alternative embodiment, the hollow tube 100 is formed by 3-D printing using a thermoplastic or photopolymerizable biocompatible material. In an embodiment with an integrally formed seal 120 within the hollow tube 100, the hollow tube 100 may be formed by over-molding blow-molded or injection-molded elastomeric material over a separately formed seal. The term integrally formed as used herein means that the components mentioned are made together as a single unit or inextricably bonded during manufacture.

The constitutive material of any element of the percutaneous transabdominal port may be impregnated or coated with antimicrobial material in an attempt to decrease infection. An antimicrobial agent may be incorporated via dispersion into uncured elastomer, or applied as an external coating. Commonly used antimicrobial agents include silver, silver alloy, or silver compounds such as silver oxide; hydrogels; or antibiotics,

Still referring to FIGS. 2A-B, the percutaneous transabdominal port further includes an internal retainer 118. The internal retainer 118 may be integrally formed with the internal end 110 of the hollow tube 100, and acts to secure the percutaneous transabdominal port within the body. When the percutaneous transabdominal port is positioned in a patient, the internal retainer 118 sits within the organ 108 in its wide configuration, as depicted in FIG. 1. In the wide configuration, the diameter of the internal retainer 118 exceeds the diameter of the abdominal wall tract 102, such that the percutaneous transabdominal port cannot be easily withdrawn into the tract and out of the body.

In some embodiments, the percutaneous transabdominal port contains a seal mechanism 120 to prevent fluid reflux from the organ. Such a seal mechanism permits passage and withdrawal of a catheter or other instrument through the percutaneous transabdominal port into the organ 108, but blocks fluid flow if no instrument is inserted. The seal 120 may repeatedly accommodate passage of an instrument without sustaining substantial damage. The seal 120 may be integrally formed with the internal retainer 118 as depicted in FIGS. 2A-B. In alternative embodiments, the seal 120 is separately formed and attached to the internal retainer 118 either with adhesive bonding or by over-molding as the internal retainer 118 is formed. The seal 120 may also be located within the channel 114 of the hollow tube 100 and any point along its length. In some embodiments, multiple seals in series may be employed.

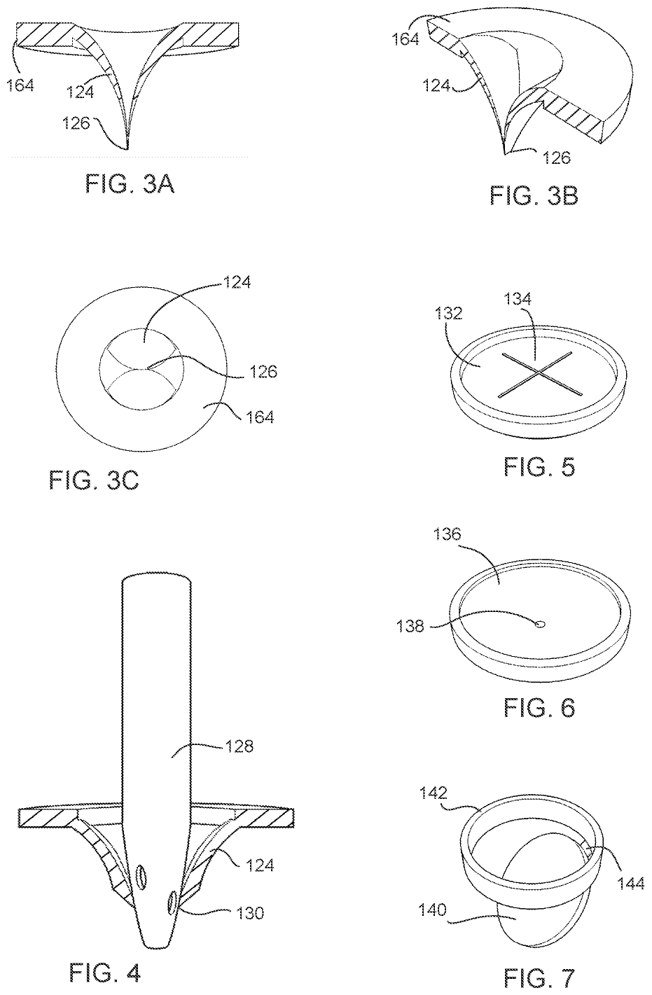

The seal 120 may block fluid flow via various mechanisms. In some embodiments, an elastomeric seal forms around inserted instruments that are passed through. The seal then coapts to create a blockage when no instrument is inserted. One such seal mechanism is a duckbill valve, depicted in FIGS. 3A-C. Such a valve has two or more leaflets 124 which are usually in a closed, coapted configuration 126. Pressure surrounding these leaflets 124, such as that from fluid within an organ, can aid in maintaining the closed configuration. The leaflets 124 separate in response to applied pressure to allow passage of an instrument 128, forming an elastomeric seal 130 around said instrument 128 (see FIG. 4). An alternative seal mechanism is a cross-slit valve (FIG. 5). Such a valve comprises an elastomeric diaphragm 132 with two or more cusps 134 that coapt at rest but open in response to applied pressure. Still another seal embodiment is a diaphragm valve (FIG. 6). In such an embodiment, the valve has an elastomeric diaphragm 136 with a small central opening 138 that limits fluid passage at rest. In response to applied pressure, the opening 138 dilates to accommodate and form an elastomeric seal around an instrument (e.g., instrument 128), then elastically returns to its original size when said instrument is removed. An alternative embodiment comprises a one-way flapper valve (FIG. 7). In such an embodiment, a flat flapper gate 140 is attached to the valve housing 142. At rest, the gate 140 lies perpendicular to directional flow. The gate 140 is attached to its housing by a unidirectional hinge 144, such that the gate 140 will rotate open in response to pressure applied from one direction but does not rotate in response to pressure from the opposite direction.

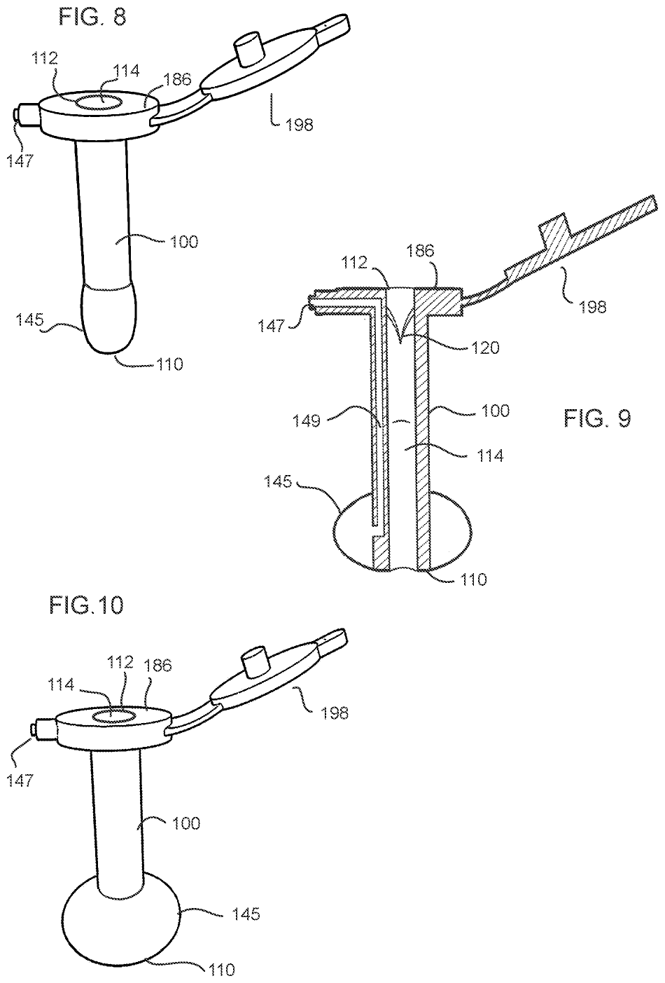

There exist various embodiments of the internal retainer 118. One embodiment includes an inflatable balloon 145 integrally formed with the internal end 110 of the hollow tube 100. Such a balloon has a thin, flexible wall. In a non-inflated state, the balloon 145 is collapsed in a first, narrow configuration that approximates the outer diameter of the hollow tube (FIG. 8) The balloon 145 is fluidly connected to an integrated check valve 147 (or other inflation means permitting inflation) at the external end 112 of the percutaneous transabdominal port via an inflation channel 149 (FIG. 9). Fluid may be instilled via the integrated check valve 147 into the inflation channel 149 and into the inflatable balloon 145 Instilled fluid distends the inflatable balloon 145, widening its diameter. Such an internal retainer thus assumes a second, wider configuration when inflated (FIG. 10).

Materials commonly used to manufacture a retention balloon for a medical device are biocompatible and include latex, silicone, polyurethane, pebax elastomers and other compliant thermoplastics. There are multiple methods of balloon manufacture including but not limited to extrusion, blow-molding, stretch blow-molding, liquid injection molding, and dip molding. There are multiple methods to attach a balloon to a hollow tube, including but not limited to glue or thermal bonding.

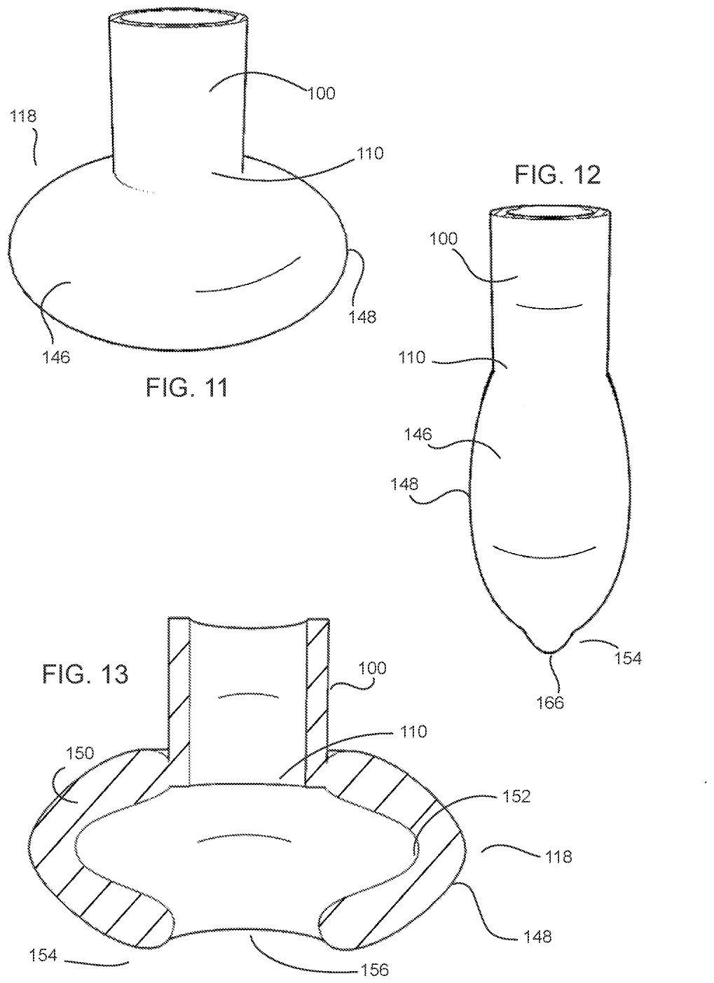

In another embodiment, the internal retainer is elastomeric and resiliently deformable. Such an internal retainer may include a deformable flange 146 (FIG. 11). The usual configuration of such an internal retainer is its wide configuration, wherein the flange diameter is larger than the diameter of abdominal wall tract 102. The internal retainer 118 may be configured in a second, narrow configuration (FIG. 12), in which the diameter of the flange 146 is decreased to approximate the diameter of the hollow tube 100. In a narrow configuration, the internal retainer 118 may pass through the abdominal wall tract 102 for insertion and removal of the percutaneous transabdominal port.

Converting the internal retainer to its narrow configuration may rely on deformation caused by a mechanical force applied to an interior surface of the internal retainer, for example by using a straightening rod 170, described below. Converting the internal retainer to its narrow configuration may also rely on deformation caused by a mechanical force applied to an outer surface 148 of the internal retainer, for example by using an introducer sheath, trocar, or capsule, described below. In an embodiment with a deformable internal retainer, such as is described in FIGS. 11, 12, “converting” the internal retainer to its wide configuration means allowing the internal retainer to relax to its static shape absent force applied to it.

A deformable internal retainer may be constructed from elastomeric, biocompatible materials, preferably shaped via injection molding. Exemplary materials include silicone, polyurethane, polyvinylchloride, and latex. In embodiments, elastomeric components of the internal retainer may be over-molded around rigid thermoplastic components.

Several examples of deformable internal retainers are illustrated. FIG. 13 depicts a cross sectional view of an embodiment of the internal retainer 118 with a bulbous flange 146. A bulbous flange 146 may be largely hollow so as to permit deformation. In general, the thickness of a bulbous flange wall 150 is between 1 millimeter and 10 millimeters. The wall thickness may be uniform or nonuniform. In a specific embodiment, the wall thickness is thinner at the deformation hinge point 152 so as to facilitate transition to a narrow configuration.

Referring now to FIGS. 13-16B, an internal retainer tip 154 may have an opening 156 to permit passage of fluid or an instrument through the tip of the internal retainer 118. In such an embodiment, it may be advantageous to have elements that guide a catheter or instrument through the opening 156. One such element may include internal sidewalls 158 which extend axially from the internal end 110 of the hollow tube 100 into the internal retainer 118. Another such element may include internal sidewalls 160 which extend axially into the internal retainer 118 from the internal retainer tip 154.

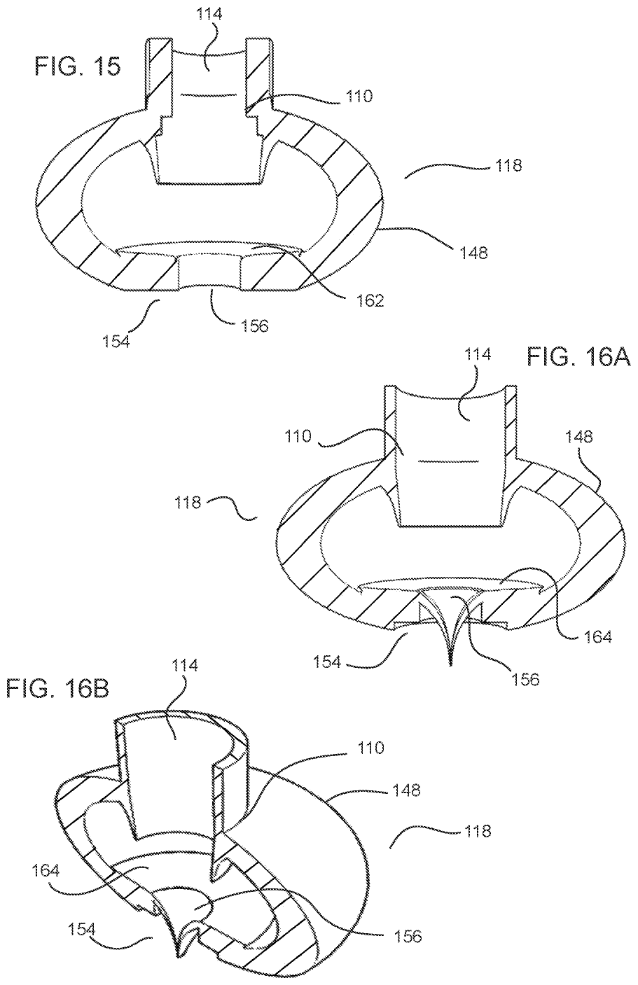

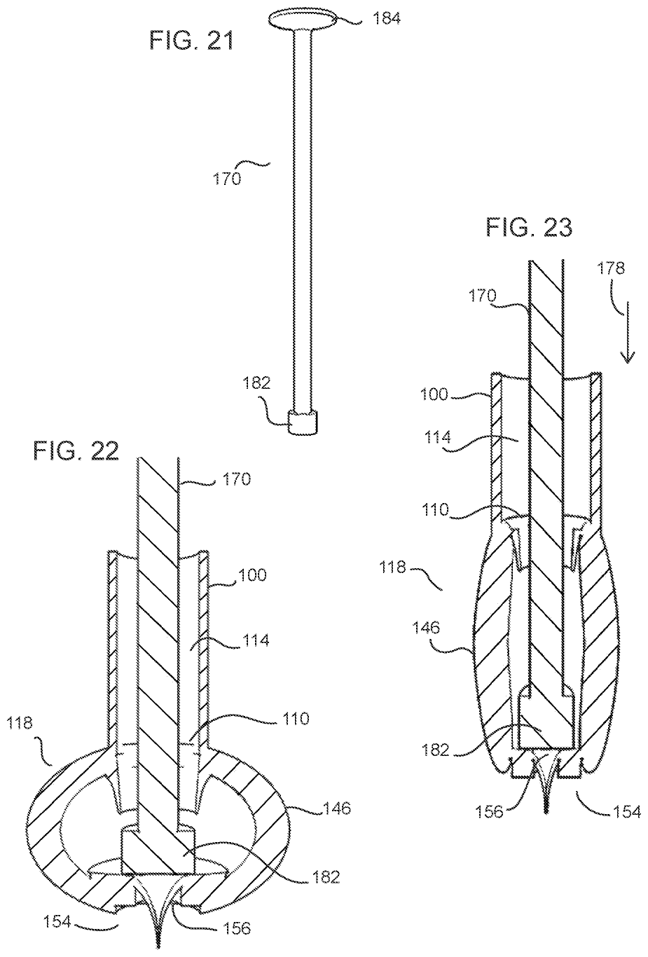

The internal retainer tip 154 may have differing designs to facilitate differing mechanisms of insertion. In one embodiment, the opening 156 in the internal retainer tip 154 is narrower than the channel 114 of the hollow tube 100 such that a straightening rod 170 (see, e.g., FIG. 21) is sized to pass through the channel 114 but not to pass through the opening 156 at the retainer tip 154. The retainer tip 154 may include additional structures such as an internal shelf 162 surrounding the opening 156 (FIG. 15). The shelf 162 provides structure that engages a straightening rod 170 and lends additional structural integrity to the retainer tip 154. The shelf 162 may include a thickened rim of elastomeric material. Alternatively, the shelf 162 may be reinforced with a rigid plastic material. In some embodiments where a flanged seal 120 such as the flanged duckbill valve depicted in FIGS. 3A-C is integrally formed with the internal retainer tip 154, the shelf 162 may include the flange 164, as depicted in FIGS. 16A-B. In such embodiments, the diameter of the straightening rod 170 is narrower than the channel 114 to allow it to pass, but larger than the internal retainer tip opening 156 such that the straightening rod 170 engages the internal retainer tip 154 and does not pass through the opening 156.

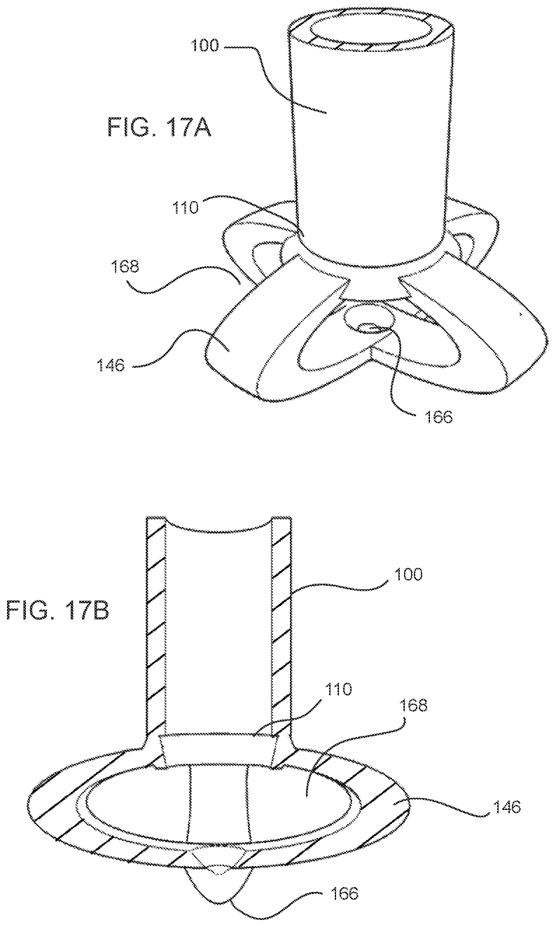

In an alternative embodiment, the internal retainer tip 154 is closed 166, as depicted in FIGS. 17A-B. In such an embodiment, apertures 168 in the sides of the retainer flange 146 allow passage of fluid or an instrument 128.

A deformable internal retainer 118 is deformed to a narrow configuration (FIG. 12) to pass through the abdominal wall tract 102. Achieving the narrow configuration may rely on insertion of a straightening rod 170 that deforms the internal retainer 118. Such a straightening rod 170 is for example made from rigid, non-elastic material such as plastic or metal. The straightening rod 170 is configured to pass through the channel 114 including the opening at the internal end 110 of the hollow tube 100. The straightening rod 170 engages the retainer tip 154 without passing through the tip opening 156. In such an embodiment, the internal retainer flange 146 is made from elastomeric material such that that it is deformable. In some embodiments, when axial pressure is applied to the internal retainer tip 154 the flange 146 deforms longitudinally such that the diameter of the internal retainer decreases as its length increases.

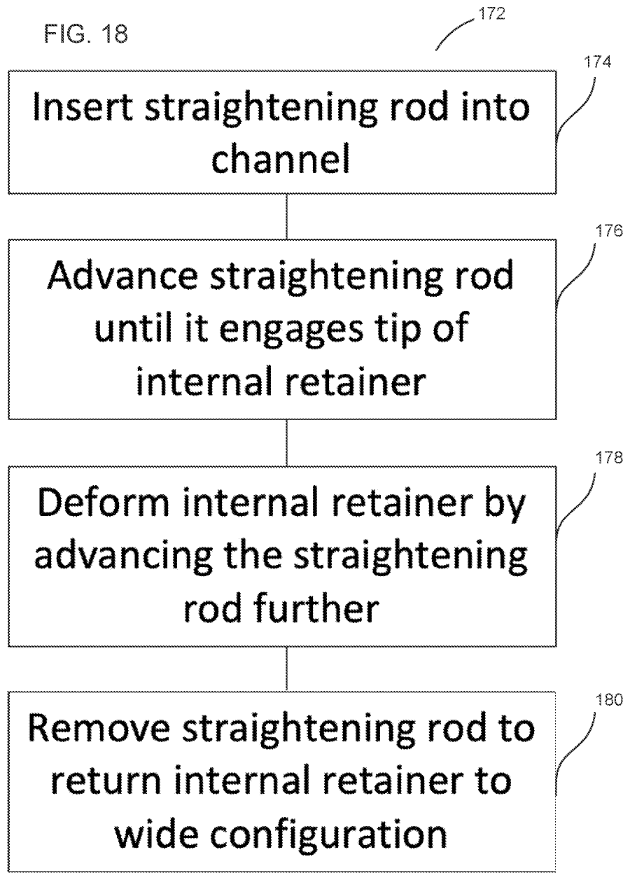

Referring now to FIG. 18, a method 172 to convert a deformable internal retainer 118 to a narrow configuration using a straightening rod 170 is disclosed. First, the straightening rod is inserted 174 through the channel 114. Next, the straightening rod 170 is advanced 176 through the internal end 110 of the hollow tube 100 and engages the internal retainer tip 154. Additional axial pressure is applied 178, longitudinally deforming the internal retainer 118 into a narrow configuration. When the straightening rod is removed 180, the internal retainer flange 146 elastically returns to its natural wide configuration.

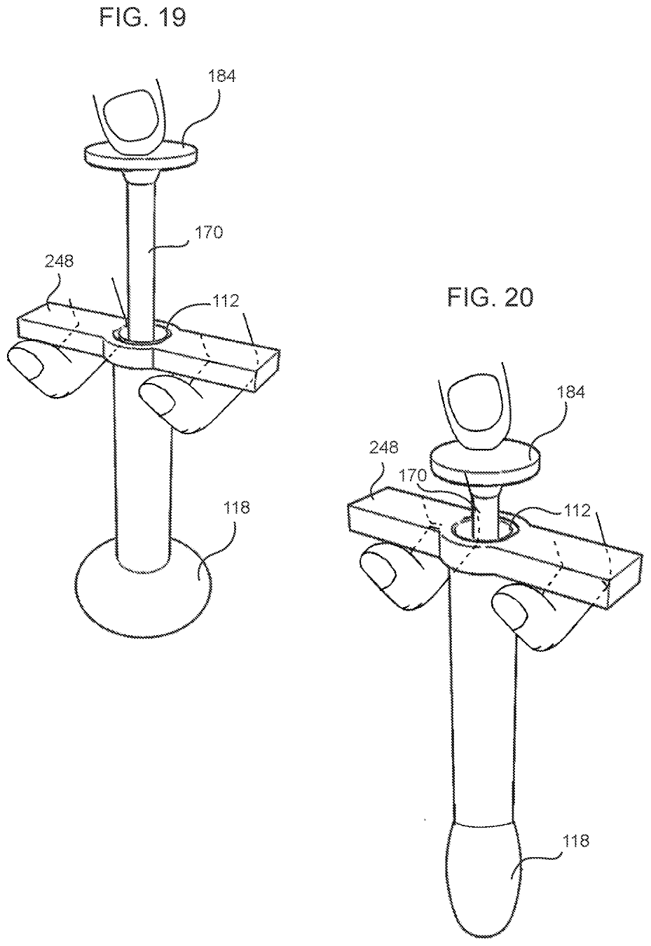

To facilitate applying axial pressure to the straightening rod 170, there may be an integrally formed platform 184 on the external end of the straightening rod 170. Installers of the percutaneous transabdominal port may hold the hollow tube 100 between their second and third fingers and place their thumb on the platform 184 to apply axial pressure. Optionally, an external retainer 186 or finger tabs 248 coupled to the external end 112 of the hollow tube 100 may facilitate holding the hollow tube 100. The external retainer flange 190 or finger tabs 248 may be engaged by the second and third fingers to hold the hollow tube 100 in place while pushing the end platform 184 of the straightening rod 170 with a thumb through the channel 114. FIGS. 19, 20 depict an embodiment of the percutaneous transabdominal port where the hollow tube 100 has integrally formed finger tabs 248 at its external end 112.

An embodiment of a deformable internal retainer 118, where the internal retainer tip 154 includes a central opening 156 that is narrower than channel 114, is depicted in FIGS. 15, 16A-B. One possible straightening rod 170 for such an embodiment is depicted in FIG. 21. The tip 182 of this straightening rod has a diameter smaller than that of the channel 114 to allow the straightening rod to pass, but larger than that of the opening 156 in the retainer tip 154 to prevent the straightening rod from passing through the opening 156. FIG. 22 depicts one straightening rod 170 engaging a retainer tip 154. FIG. 23 depicts a narrow configuration of the internal retainer 118 after axial pressure has been applied to the straightening rod 170.

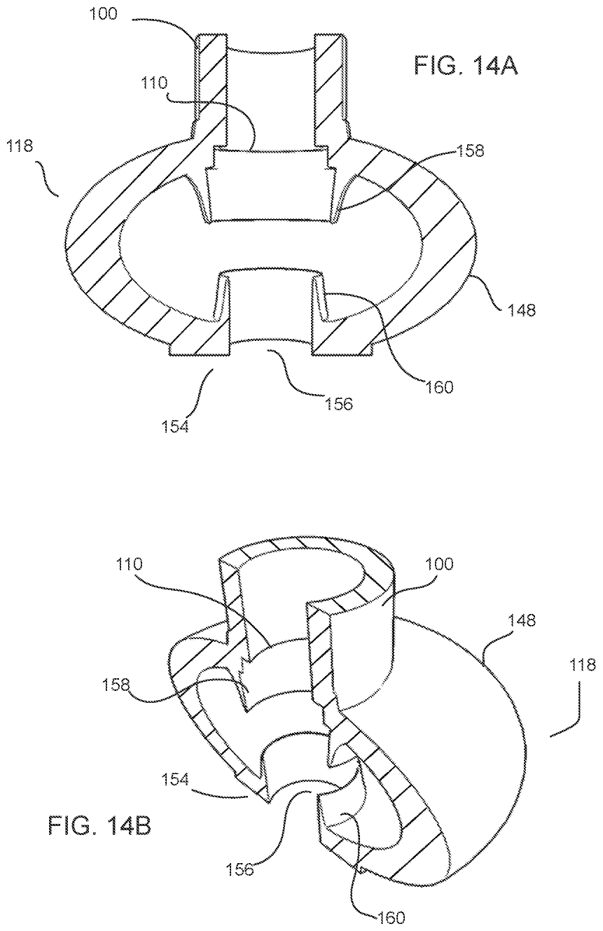

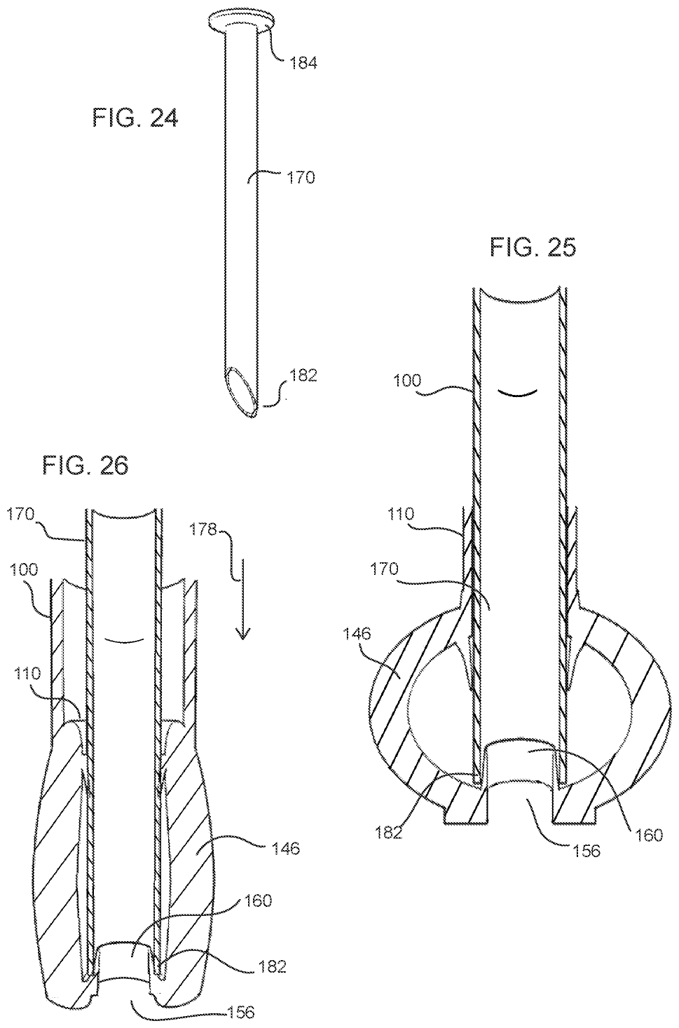

An embodiment of a deformable internal retainer 118 in which internal sidewalls 160 extend from the retainer tip 154 is depicted in FIGS. 14A-B. A straightening rod 170 for such an embodiment may engage these sidewalls 160 by surrounding them. The tip 182 of such a straightening rod may have a central opening which accommodates the internal sidewalls 160, as shown in FIG. 24. The straightening rod tip 182 seats circumferentially around the internal sidewalls 160 to engage the retainer tip 154, as shown in FIG. 25. FIG. 26 depicts a narrow configuration of the internal retainer 118 after axial pressure has been applied to the straightening rod 170 in this embodiment.

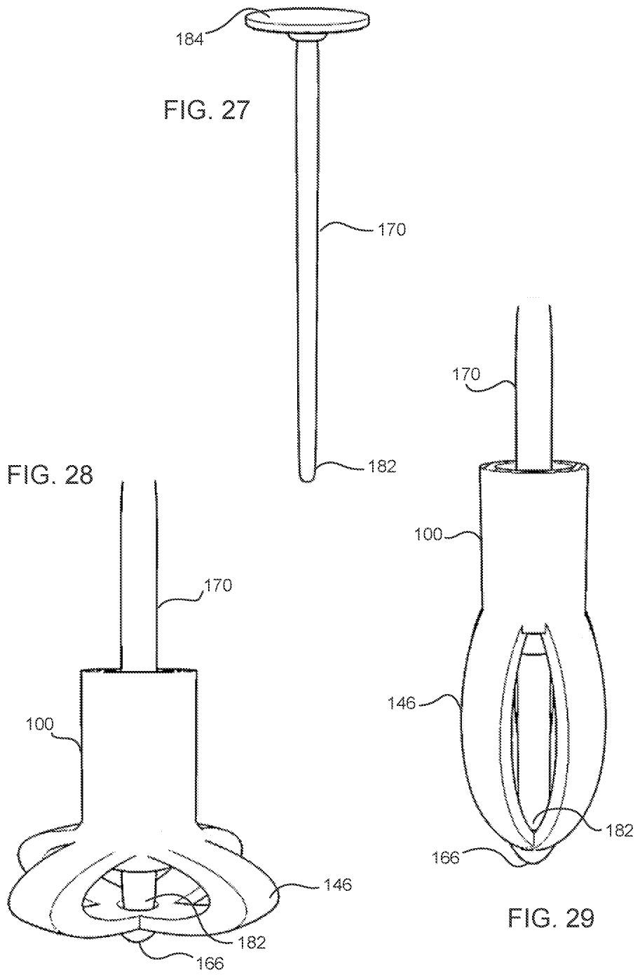

Referring now to an embodiment of a deformable internal retainer with a closed tip 166 as depicted in FIGS. 17A-B. A straightening rod 170 for this embodiment engages the closed tip 166. The straightening rod 170 for this embodiment may be mandrin-like as depicted in FIG. 27. Such a straightening rod 170 may be narrow, and may be made of a metal such as stainless steel. The straightening rod tip 182 may engage the closed internal retainer tip 166, as depicted in FIG. 28. FIG. 29 depicts a narrow configuration of the internal retainer 118 after axial pressure has been applied to the straightening rod 170 in this embodiment.

In some embodiments, a deformable internal retainer 118 may be converted from its natural wide configuration to a narrow configuration by applying mechanical force to an outer surface 148 of the deformable internal retainer 118.

For example, the percutaneous transabdominal port may be entirely or partially placed within a peel-away introducer sheath (not pictured). The introducer sheath surrounds the internal retainer 118, deforming the flange 146 to its narrow configuration. The percutaneous transabdominal port in its narrow configuration within the surrounding sheath may be passed through the abdominal wall tract 102. The sheath may be removed, which removes the mechanical force exerted on the outer surface 148 of the internal retainer 118 allowing it to elastically return to its wide configuration.

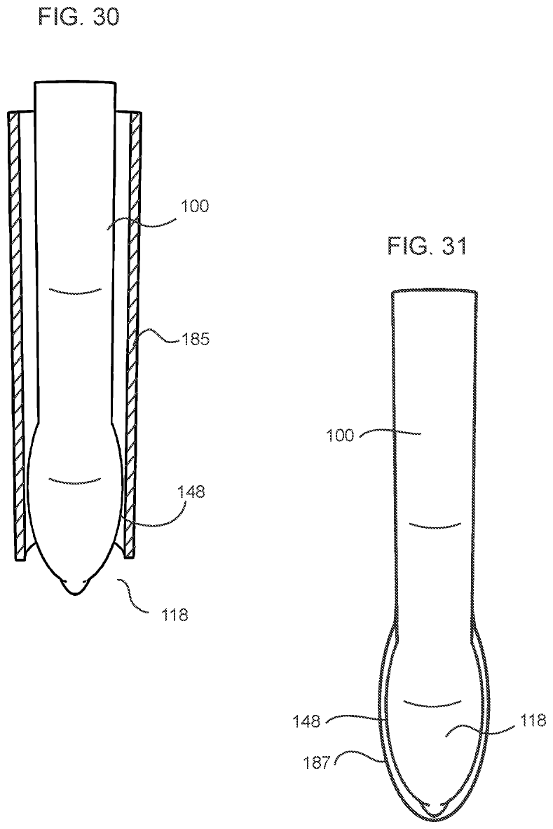

Similarly, the percutaneous transabdominal port may be placed within a trocar 185 (FIG. 30), which is passed through an abdominal wall tract 102. Once the tip of the trocar 185 is within the organ 108, the percutaneous transabdominal port may be deployed through the trocar 185 into the organ 108 via a plunger. After the percutaneous transabdominal port is deployed through trocar 185, the trocar no longer exerts mechanical force on the outer surface 148 of the internal retainer 118. The internal retainer 118 thus elastically returns to its natural wide configuration. The trocar 185 may then be withdrawn.

Alternatively, the deformable internal retainer 118 may be placed in a narrow configuration within a soluble capsule 187 (FIG. 31). Such a capsule may dissolve upon contact with fluid within the organ 108, releasing the deformable internal retainer which then elastically returns to its wide configuration. Such a capsule may be constructed from biodegradable materials including gelatin, hydrolyzed starch, or cellulose.

The percutaneous transabdominal port further includes an external retainer 186. The external retainer 186 couples with the hollow tube 100 external to the body. When the percutaneous transabdominal port is positioned in a patient, the external retainer 186 rests against the external surface of the abdomen 106.

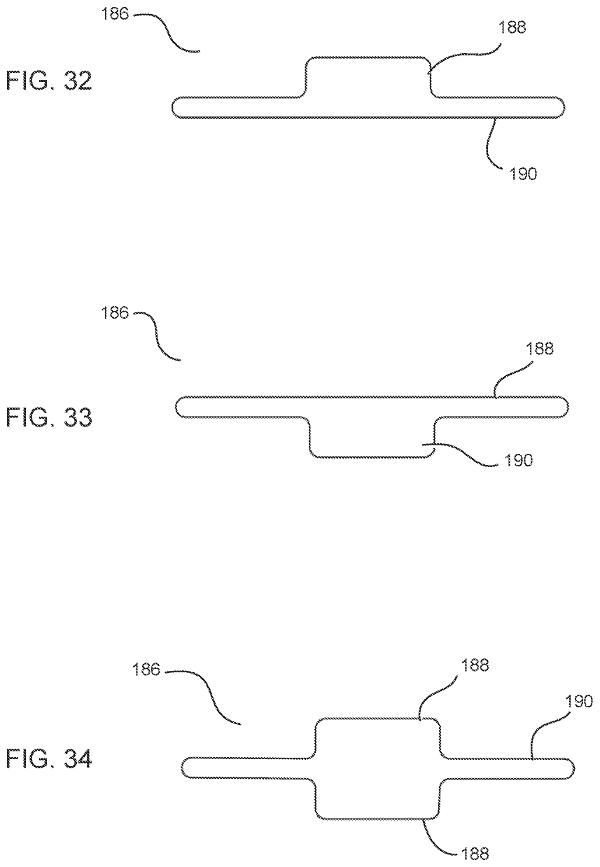

The external retainer 186 may have two elements: a collar 188 which surrounds the hollow tube 100 and a flange 190 which acts as a retention bolster. The collar 188 is for example tube-shaped and engages the hollow tube 100 within its central opening 192. The collar 188 may have various embodiments. The length of the collar 188 is variable and can be as narrow as the thickness of the flange. In general, the length of the collar 188 is between 2 millimeters and 30 millimeters. If the length of the collar 188 exceeds the thickness of the flange 190, the collar 188 extends coaxially along the hollow tube 100. The collar 188 may for example extend upward (FIG. 32), downward (FIG. 33), or both upward and downward along the hollow tube 100 (FIG. 34).

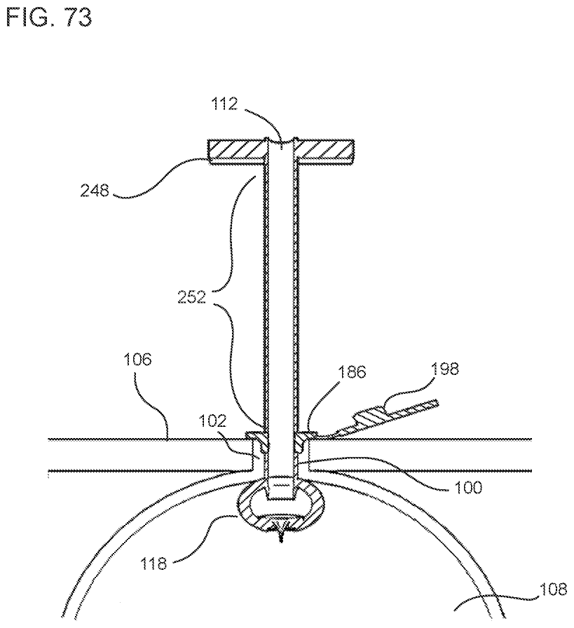

The wall thickness of the collar 188 may be uniform or nonuniform. Additionally, the collar 188 may be tapered. In one embodiment, a tapered collar 188 extending downward toward the abdominal wall 104 may partially insert into the abdominal wall tract 102, as depicted in FIGS. 73-75. Such an embodiment may decrease urine leakage from the abdominal wall tract, or may allow the external retainer to have a lower profile.

The flange 190 of the external retainer 186 may have various embodiments. In all embodiments, the diameter of the flange 190 exceeds the diameter of the abdominal wall tract 102 such that it prevents retraction of the percutaneous transabdominal port into the body. In general, the diameter of the flange 190 exceeds the diameter of the external end 112 of the hollow tube 100 by 5 millimeters to 50 millimeters. In general, the thickness of the flange 190 is between 1 millimeter and 10 millimeters. The flange is preferably flexible. It may be constructed from biocompatible elastomeric materials such as silicone, polyurethane, polyvinylchloride, or latex.

The flange 190 may form apertures 194. Such apertures may serve to allow air flow between the external abdominal surface 106 and the percutaneous transabdominal port. Alternatively, such apertures 194 may serve to accommodate sutures to secure the flange to the external abdominal surface 106.

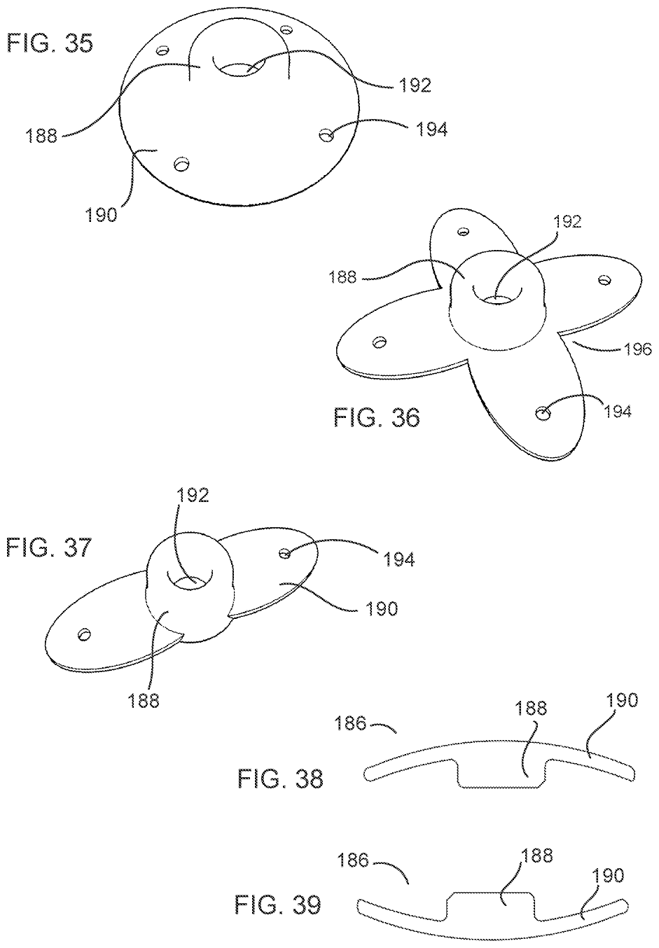

In some embodiments, the shape of the flange 190 can be substantially contiguous, as depicted in FIG. 35. Alternatively, the flange can be notched 196 to form petals (FIG. 36) or discontinuous to form wings (FIG. 37). Moreover, the contour of the flange 190 may vary. The flange 190 may be substantially flat as it engages the external surface of the abdomen 106 (FIGS. 32-34). Alternatively, the flange 190 may extend from the external retainer collar 188 in a convex (FIG. 38) or concave (FIG. 39) curved configuration. Such configurations may be advantageous; for instance, a convex or “dome-like” configuration yields open space housed between the external retainer 186 and the external abdominal surface 106. Such a space could be used to accommodate an absorbent and/or antibiotic-impregnated pad.

In some embodiments, the external retainer 186 may be integrally formed with the external end 112 of the hollow tube 100. In alternative embodiments, the external retainer may be assembled with the external end 112 of the hollow tube 100. In embodiments requiring assembly, it may be advantageous for the external retainer 186 to couple securely to the hollow tube 100 such that, once positioned, it remains anchored in place and does not uncouple from the hollow tube 100.

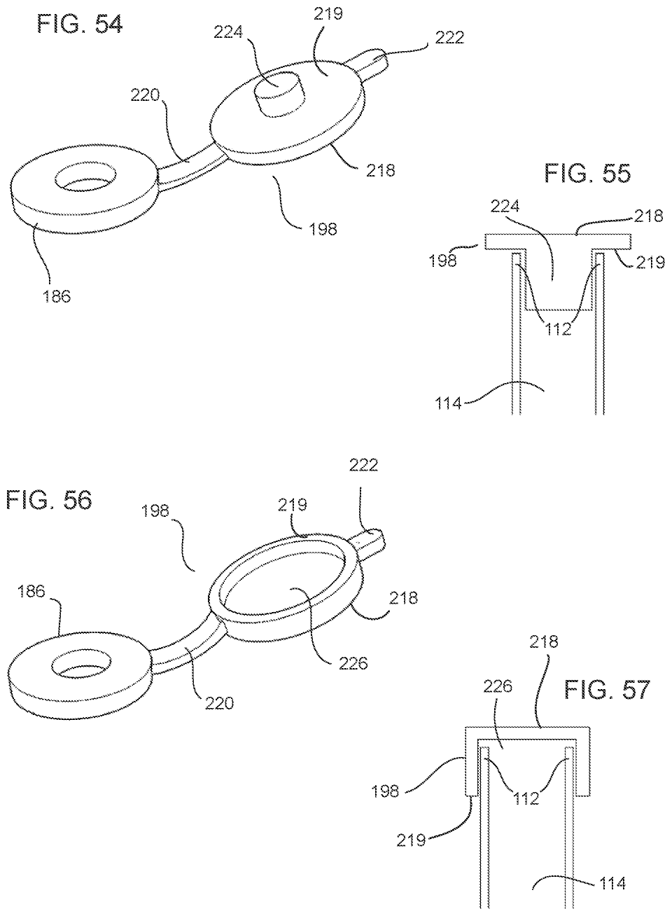

Various methods may be used to secure the external retainer 186 to the hollow tube 100. One embodiment employs friction between the inner surface of the external retainer collar 188 and the outer surface 122 of the hollow tube 100. In such an embodiment, the diameter of the opening 192 in the external retainer is minimally larger than the external diameter of the hollow tube 100 to ensure a snug fit. Friction may be augmented using biocompatible adhesive. Friction may also be augmented by texturing the inner surface of the external retainer collar 188 and/or the outer surface of the hollow tube 100, for instance with protrusions, ridges, or grooves. Friction may also be augmented by a plug-like external closure cap 198 when in the closed configuration. In this embodiment, a plug 224 inserted into the channel 114 of the hollow tube 100 at its external end 112 fits snugly and exerts outward pressure, trapping the hollow tube wall 116 between the external retainer collar 188 and the plug 224 of the closure cap 198. Such a closure cap 198 is described and illustrated in connection with FIGS. 54, 55.

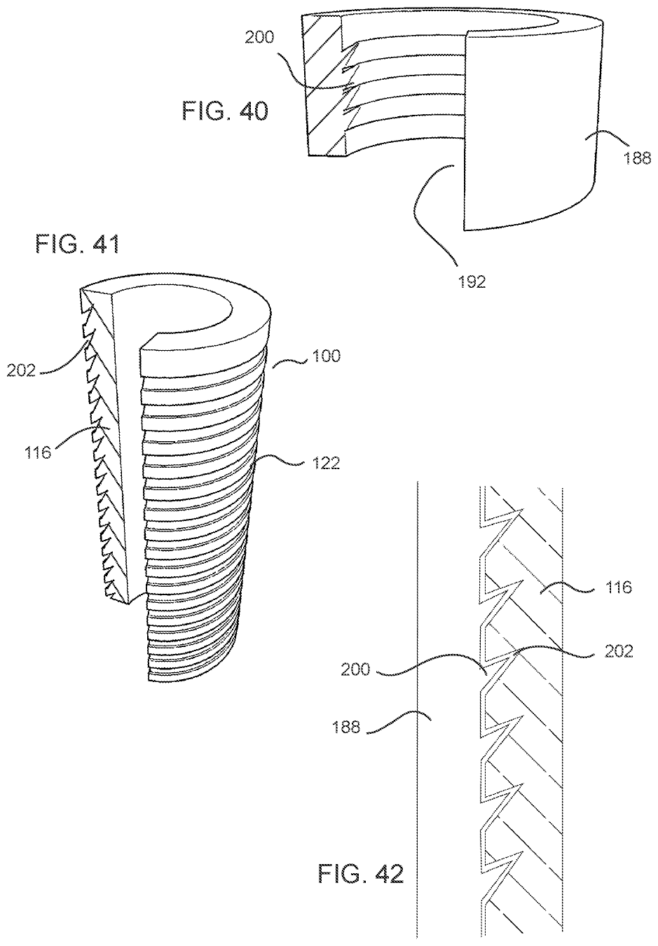

In an embodiment, securing the external retainer 186 to the hollow tube 100 employs a one-way ratchet. Such a ratchet may rely on teeth or protrusions 200 into the central opening 192 of the external retainer collar 188 (FIG. 40) engaging corresponding grooves 202 in the outer surface 122 of the hollow tube 100 (FIG. 41). In one such embodiment, the outer surface 122 of the hollow tube 100 has circumferential, angled grooves 202 within the hollow tube wall 116 (FIG. 41). The grooves 202 may be narrowly spaced for fine adjustment and positioning of the external retainer 186. The external retainer collar 188 has corresponding circumferential angled protrusions 200 into its central opening 192. The angled nature of the protrusions 200 and grooves 202 serve to permit unidirectional coaxial sliding of the external retainer 186 along the hollow tube 100 toward the surface of the abdomen 106. In this embodiment therefore, the angled protrusions 200 of the external retainer 186 engage with the angled grooves 202 of the hollow tube 100 such that the retainer cannot slide away from the abdominal surface 106. FIG. 42 depicts a profile of the protrusions 200 on the external retainer collar 188 engaged with the grooves 202 in the hollow tube wall 116.

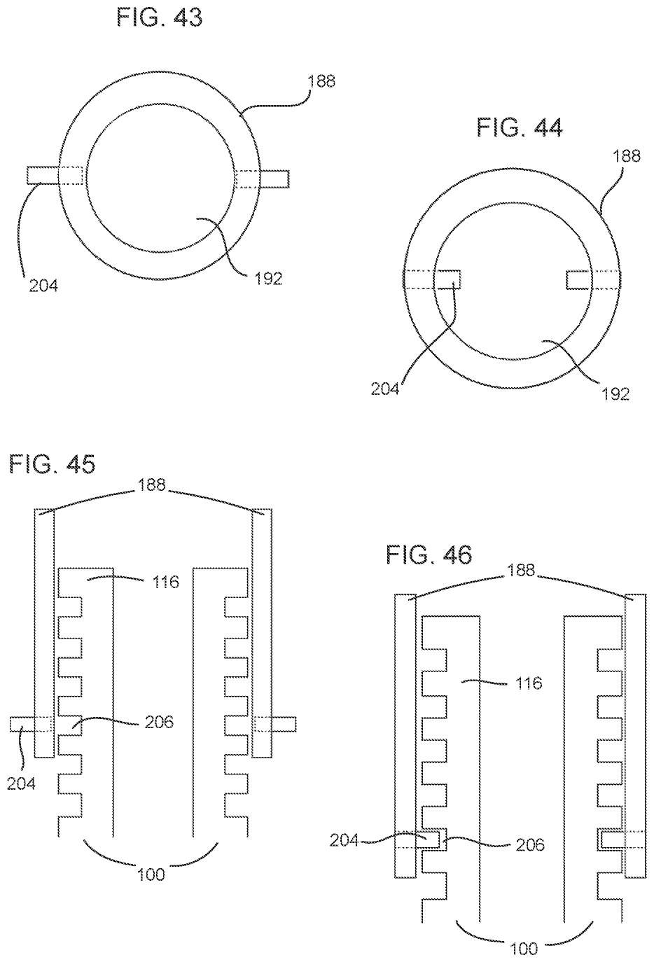

Certain embodiments employ a pushpin locking mechanism to secure the external retainer 186 to the hollow tube 100. For example, pushpins 204 may pass through the external retainer collar 188 to engage a depression 206 in the hollow tube wall 116. FIG. 43 shows a top view of the external retainer collar 188 with the pushpins 204 in an open position. FIG. 44 shows the pushpins 204 pushed inward toward the external retainer opening 192 into their closed, locked position. FIGS. 45-46 show a profile view of the external retainer collar 188 in this embodiment surrounding the hollow tube 100. The hollow tube 100 may thus have series of depressions 206 on its outer surface 122 to accept the pushpins 204. The depressions 206 are for example narrowly spaced to allow for fine positional adjustments. FIG. 45 depicts an open configuration of the pushpins 204 as the external retainer collar 188 slides along the hollow tube 100. FIG. 46 depicts the pushpins 204 in the locked position, deployed into a corresponding depression 206 in the hollow tube wall 116, anchoring the retainer 186 to the hollow tube 100.

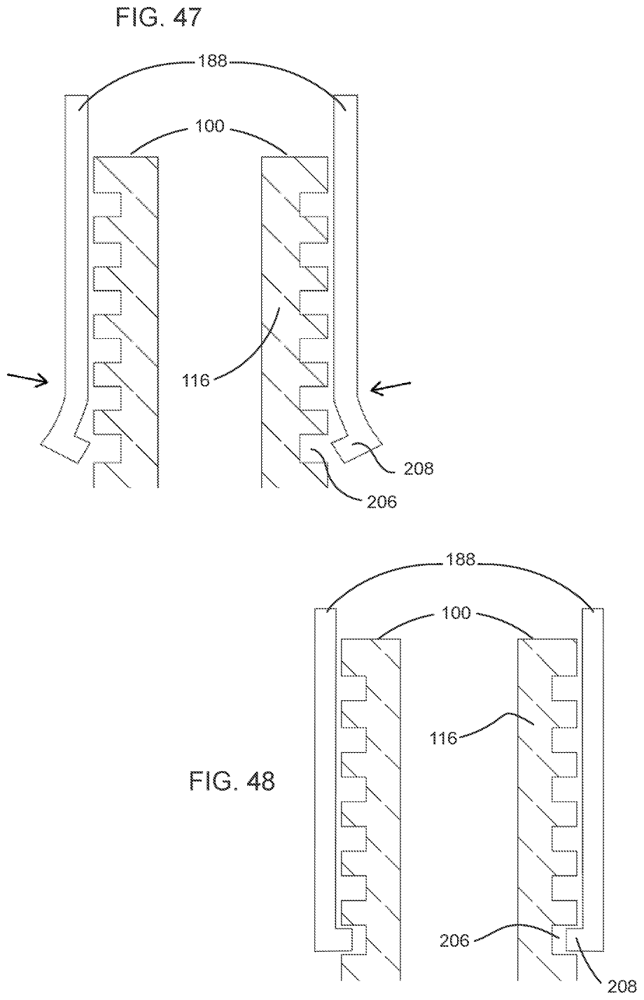

In certain embodiments, a snap-fit locking mechanism is employed. In one example of such a snap-fit locking mechanism, a protrusion from the external retainer collar 188 may be actively or passively deflected during assembly and positioning. This protrusion then catches in a mating feature (such as a depression) on the outer surface 122 of the hollow tube 100, locking the external retainer 186 in place. FIGS. 47-48 depict one example of a snap-fit locking mechanism. Protruding tabs 208 connected to the external retainer collar 188 are deflected by squeezing the external retainer 186 while sliding it along the hollow tube 100. Once the external retainer 186 is positioned, pressure is released, and the tabs 208 straighten to engage a corresponding depression 206 in the hollow tube wall 116, anchoring the external retainer 186 to the hollow tube 100.

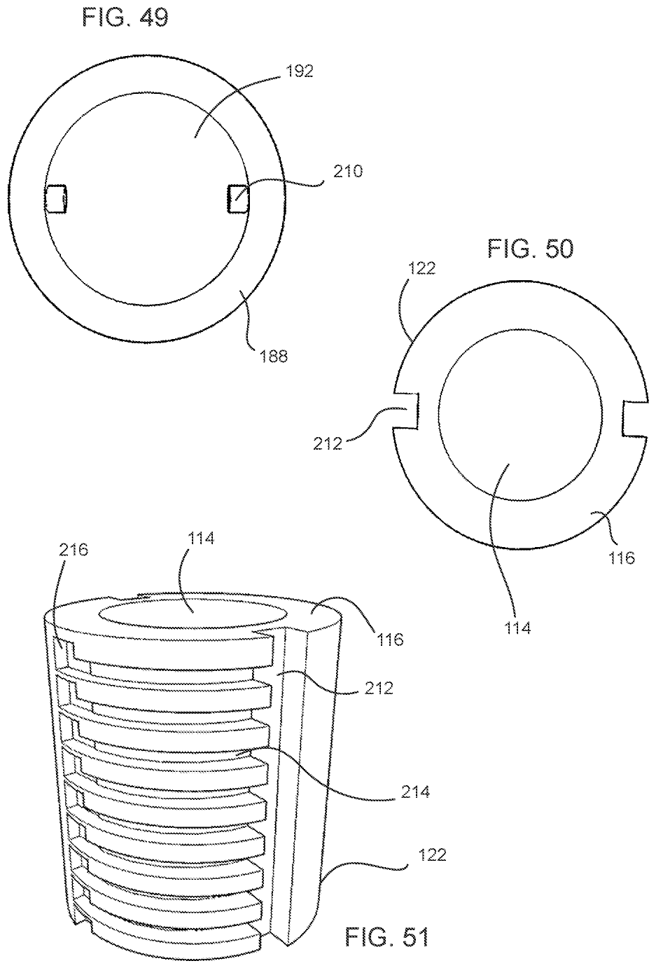

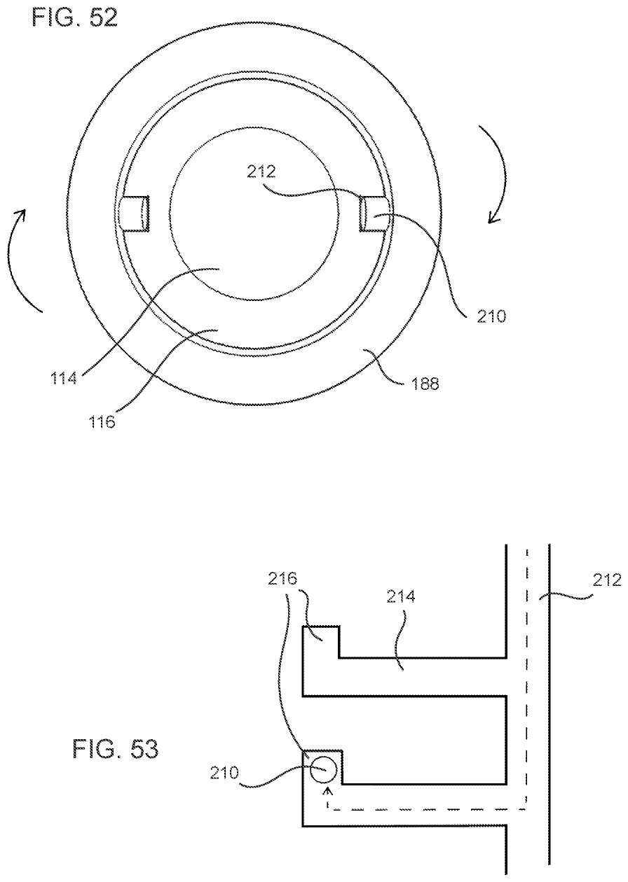

In an alternative embodiment, a twist locking mechanism is employed. FIGS. 49-53 depict an embodiment of one such mechanism. In such an embodiment, protrusions 210 on the internal surface of the external retainer fit within corresponding grooves in the hollow tube wall 116. Longitudinal grooves form slots 212 along the length of the hollow tube 100. The protrusions 210 on the external retainer collar 188 engage these slots 212 and the external retainer 186 can be advanced along the hollow tube 100 toward the abdominal surface 106. There are additional semi-circumferential grooves 214 along the length of the hollow tube 100. When the external retainer is appropriately positioned, it may be twisted such that the protrusions 210 engage said semi-circumferential grooves 214. In some embodiments, there is an additional slot 216 projecting from the tip of the semi-circumferential groove 214 that is not in line with said groove. In this embodiment, the protrusion 210 engage this locking slot 216 in the final position to anchor the external retainer 186 to the hollow tube 100 (FIG. 53).

In an embodiment, the external retainer 186 is further secured in place by suturing of the flange 190 to the skin on the patient's external abdominal wall 106. Certain embodiments of the external retainer 186 may thus include apertures 194 in the flange 190 to facilitate suturing (FIGS. 35-37).

As noted earlier, the percutaneous transabdominal port may include a closure cap 198. FIGS. 54-57 show examples of a flip cap configuration. In such embodiments, the closure cap 198 is coupled to the external retainer 186 (FIGS. 54, 56), or directly to the external end 112 of the hollow tube 100 (not pictured) via a flexible hinge 220. A tab 222 integrally formed with the cap 198 facilitates opening of the cap 198 in order to use the percutaneous transabdominal port. The closure cap 198 may include a top surface 218 and a bottom surface 219. In embodiments, a projection 224 on the bottom surface 219 of the cap 198 seats within the channel 114 of the hollow tube 100 at its external end 112, to act as a plug (FIG. 55). In another embodiment, the bottom surface 219 of the cap 198 is recessed 226 to accommodate the external end 112, covering and thereby closing the channel 114 (FIG. 57).

The external retainer 186 and closure cap 198 may be constructed from biocompatible elastomeric materials, preferably shaped via extrusion or injection molding. They may additionally be constructed from plastic or fluoroplastic material. Rigid elements such as locking mechanisms are particularly suited for plastic construction.

The external retainer 186, closure cap 198, and external end 112 of the hollow tube 100 together may be considered the “external portion” of the percutaneous transabdominal port. The external portion may be configured to couple to various standard or proprietary medical accessory devices such as drainage tubing, infusion tubing, or syringes. Examples of standard medical connectors include Luer connectors and frustoconical fittings.

In embodiments, a hollow tube 100, internal retainer 118, and external retainer 186 with optional closure cap 198 are integrally formed. Such an embodiment is disclosed in EXAMPLE 1 below.

In embodiments, a hollow tube 100 and internal retainer 118 are integrally formed. Separately, an external retainer 186 may be integrally formed with an optional closure cap 198. Such an embodiment is disclosed in EXAMPLE 2 below.

EXAMPLE 1: In this example, a hollow tube 100, internal retainer 118, external retainer 186 and closure cap 198 are integrally formed as depicted in FIG. 8. When the external retainer 186 is integrally formed with the hollow tube 100, the percutaneous transabdominal port may not be shortened. Instead, the overall length of the percutaneous transabdominal port must be selected based on the particular thickness of the patients' abdominal wall 104.

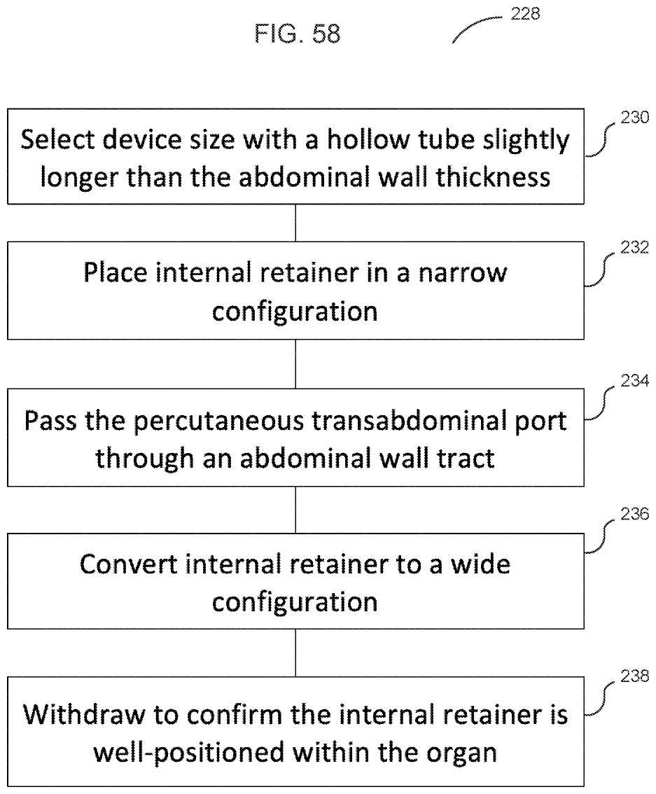

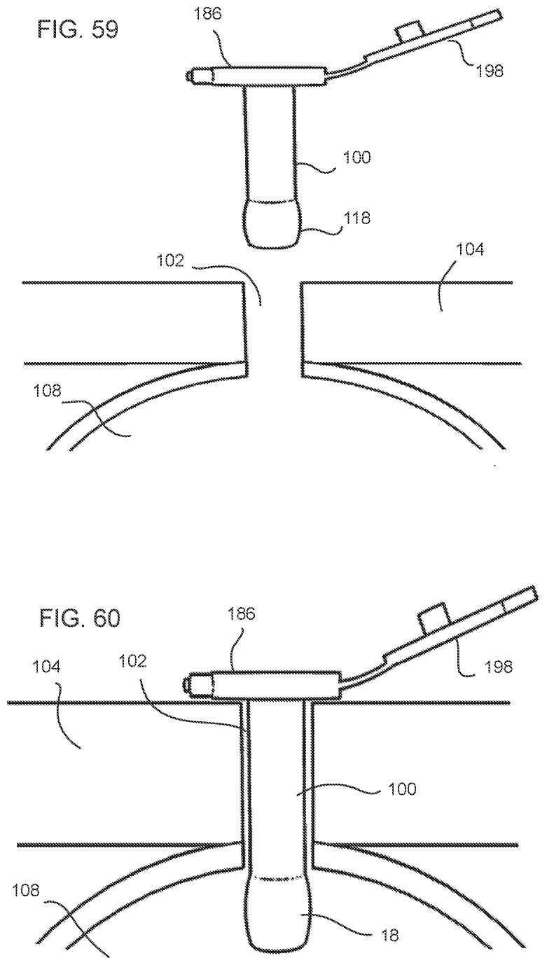

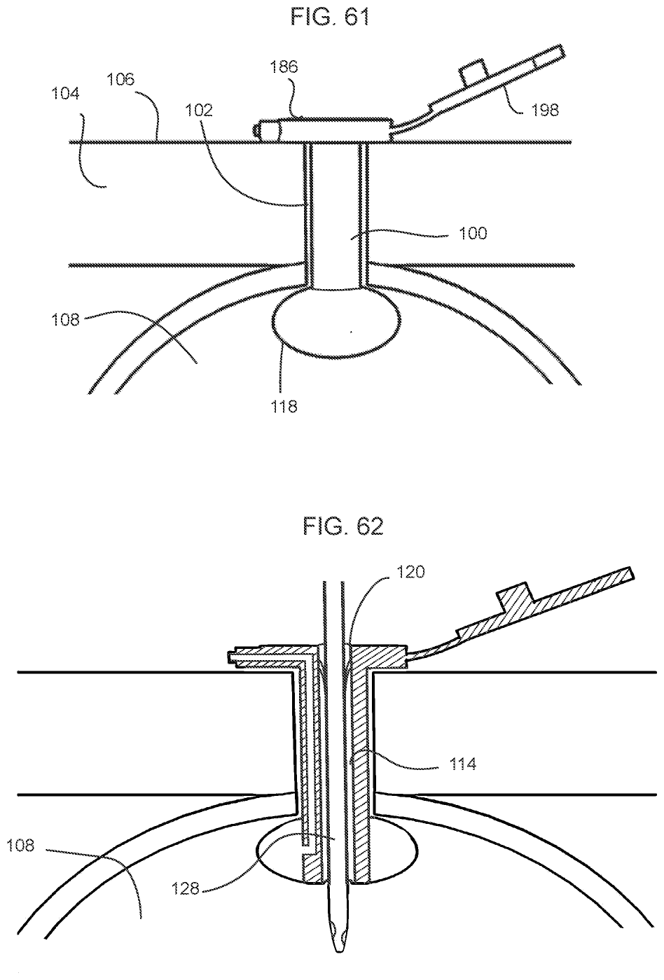

A method of insertion 228 for such an embodiment is disclosed in FIG. 58. First, a percutaneous transabdominal port of appropriate length is selected 230. The hollow tube 100 should be slightly longer than the abdominal wall thickness 104 to ensure the internal retainer 118 reaches the inside of the organ 108, as shown in FIG. 59. The internal retainer is in a deflated state or deformed 232 such that it is in a first, narrow configuration (FIG. 59). The percutaneous transabdominal port in its narrow configuration is passed 234 through the abdominal wall tract 102 and fully advanced such that the internal retainer 118 is positioned within the organ 108 (FIG. 60). The internal retainer is then converted 236 to a second, wide configuration (FIG. 61). Specifically, an inflatable internal retainer 118 may be inflated. A deformable internal retainer 118 may have its deforming mechanical force removed, allowing it to elastically return to its wide configuration. The percutaneous transabdominal port may be gently withdrawn 238 to confirm the internal retainer 118 is deployed. A correctly deployed internal retainer 118 will abut the interior surface of the organ 108 where it joins the abdominal wall tract 102 and prevent withdrawal of the percutaneous transabdominal port. Once installed, a percutaneous transabdominal port may have an instrument 128 passed through the channel 114 to access an organ 108 (FIG. 62).

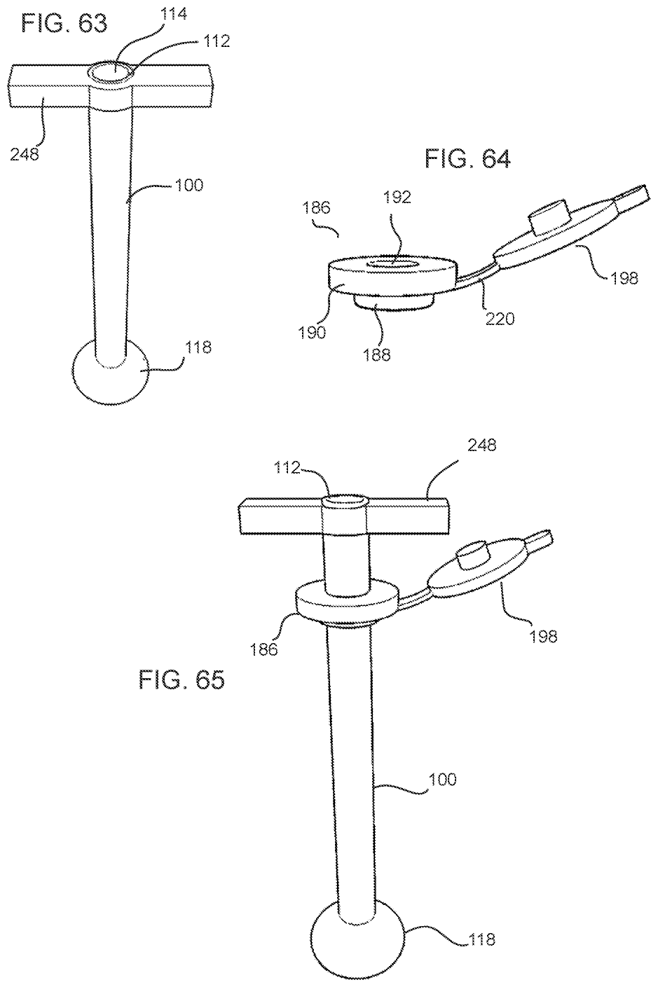

EXAMPLE 2: In this example, the hollow tube 100 and internal retainer 118 are integrally formed as shown in FIG. 63. Optionally, there are integrally formed finger tabs 248 at the external end 112 of the hollow tube 100 to facilitate ergonomic insertion. Separately, the closure cap 198 and external retainer 186 are integrally formed, as shown in FIG. 64. FIG. 65 depicts the closure cap 198 and external retainer 186 of FIG. 64 placed around the hollow tube 100.

When the external retainer 186 and closure cap 198 are not integrally formed with the hollow tube 100, shortening of the percutaneous transabdominal port is possible. The percutaneous transabdominal port of this embodiment may therefore be customized and sized based on the thickness of a patient's abdominal wall 104. Importantly, in such an embodiment, the internal retainer 118 may not rely on inflation. Shortening the hollow tube 100 as described below disrupts the inflation channel 149. Disruption of the inflation channel 149 leads to deflation of the balloon 145, and may preclude the option of shortening the hollow tube 100 to adjust its length for a particular patients' abdominal wall thickness.

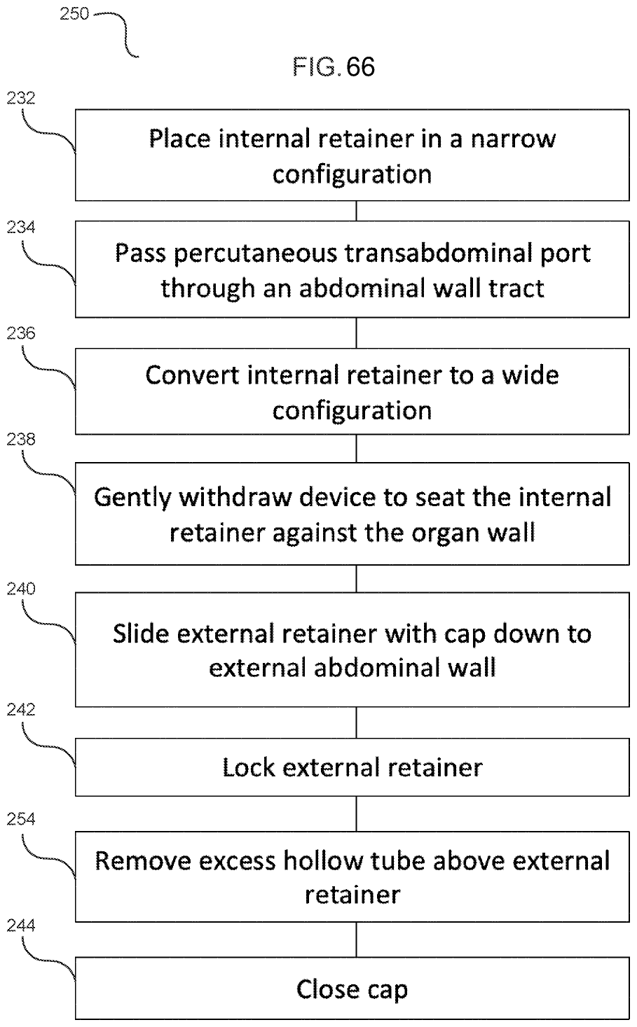

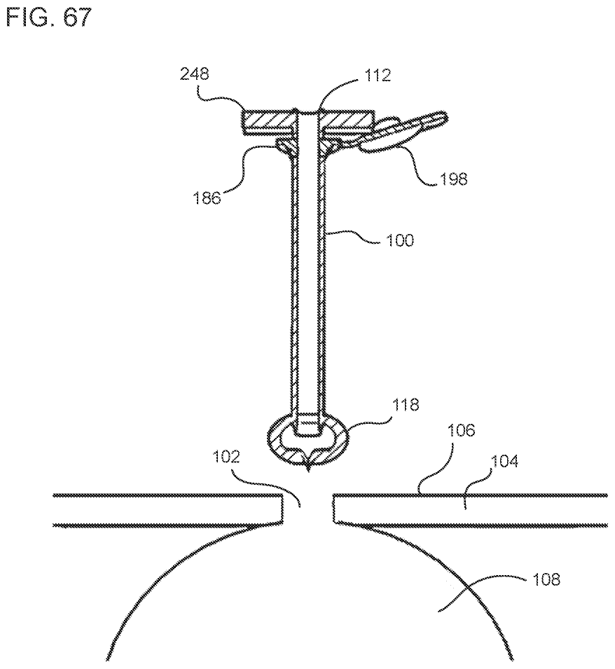

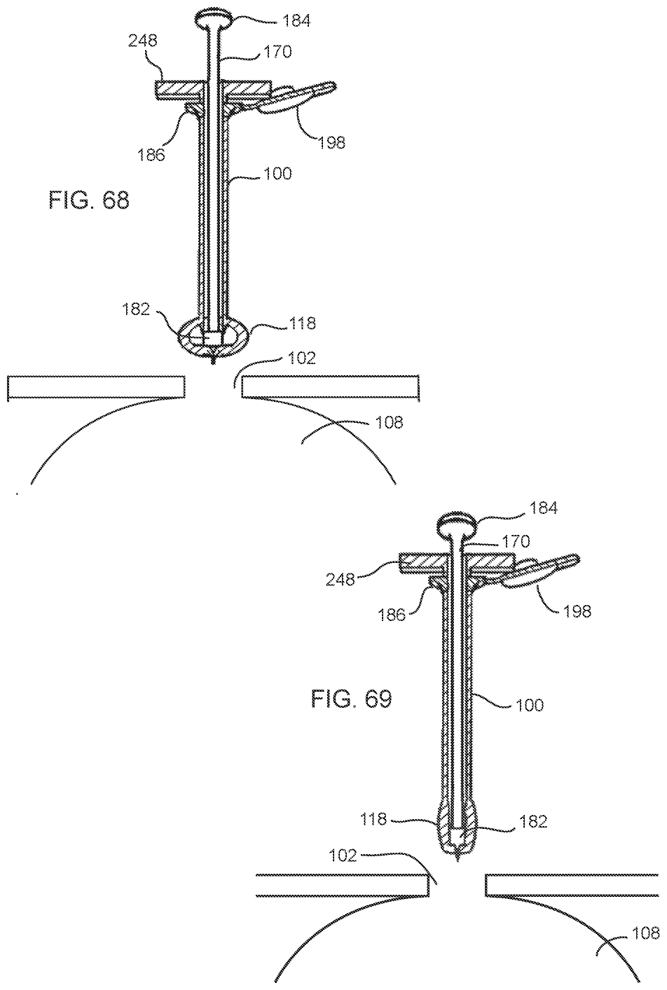

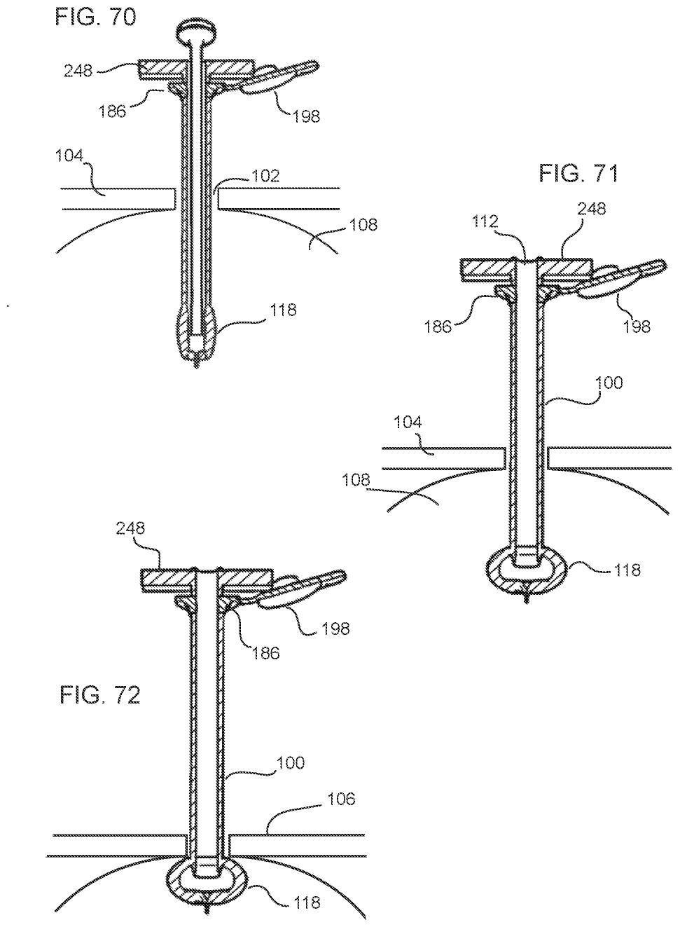

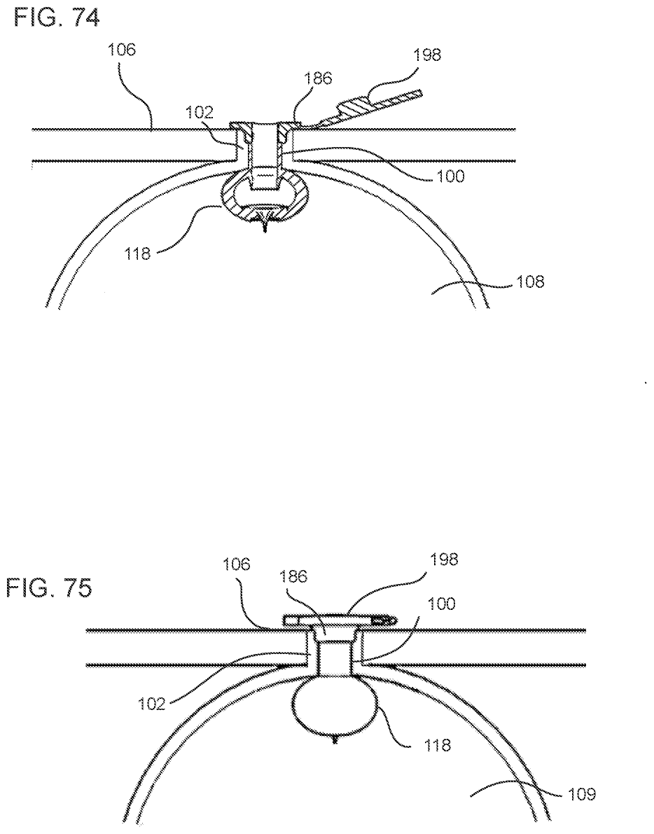

A method of insertion 250 for such an embodiment is disclosed in FIG. 66. FIG. 67 depicts a percutaneous transabdominal port outside the body prior to insertion. The internal retainer 118 is converted to its narrow configuration, for example by using a straightening rod (FIG. 68-69). The deformable internal retainer in its narrow configuration is passed 234 through the abdominal wall tract (FIG. 70) and advanced such that the internal retainer 118 is positioned within the organ 108. The internal retainer 118 is then converted 236 to its wide configuration, for example by removing a straightening rod and allowing the deformable internal retainer 118 to elastically return to its wide configuration as illustrated in FIG. 71. The percutaneous transabdominal port may be gently withdrawn 238 to confirm the internal retainer 118 is deployed inside the organ 108, and to position it against the interior surface of the organ 108 where it joins the opening of the abdominal tract 102 (FIG. 72). Next, the external retainer 186 with the integrally formed closure cap 198 are advanced 240 along the length of the hollow tube 100 until the external surface of the abdomen 106 is engaged (FIG. 73). This secures the percutaneous transabdominal port between the internal retainer 118 and the external retainer 186 and prevents motion of the percutaneous transabdominal port within the tract 102. If the external retainer relies on active locking, such as a twist lock, it is then locked 242 into place. With the external retainer 186 securely attached to the hollow tube 100, the excess tube length 252 may be removed 254 above the level of the external retainer 186. One method to remove excess hollow tube may include cutting with a bladed device such as scissors, scalpel, or guillotine device. In some embodiments, the hollow tube 100 has narrowly spaced grooves 202 to allow for precise sizing as depicted in FIG. 41. Such grooves 202 facilitate precise cutting. In some embodiments, grooves, notches, or perforations along the length of the hollow tube 100 may weaken the hollow tube such that when bent or twisted with adequate force, the hollow tube snaps off at the grooved, notched, or perforated hinge point. FIG. 74 depicts the inserted percutaneous transabdominal port after excess hollow tube 252 has been removed. The cap 198 is then closed 244 to seal the channel 114 while the percutaneous transabdominal port is not in use (FIG. 75).

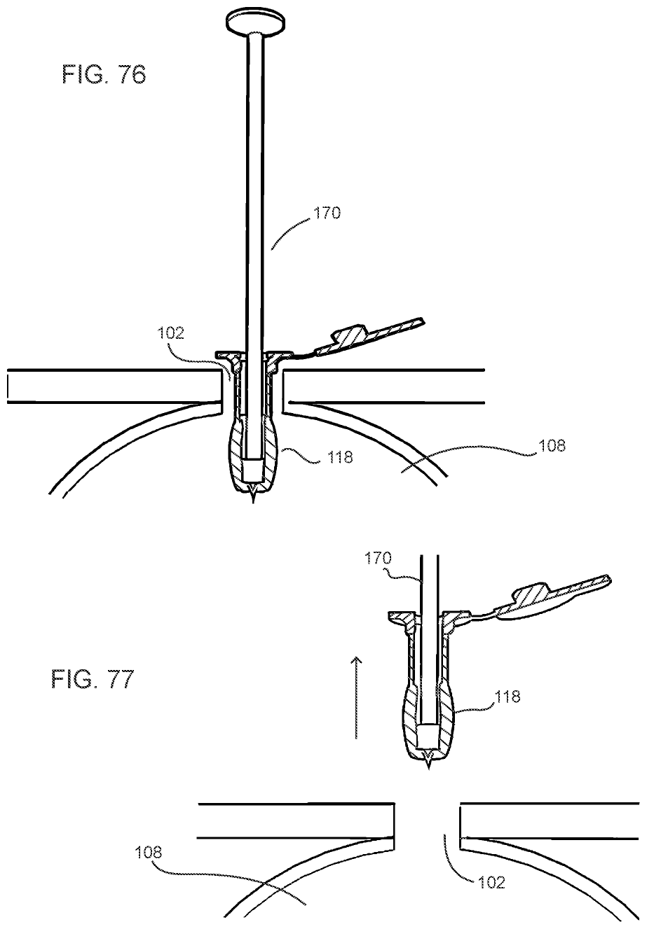

A method of removal is disclosed. The internal retainer 118 is converted to its narrow configuration. For example, an inflatable balloon 145 may be deflated. Alternatively, a straightening rod 170 is inserted through the channel 114 to achieve a narrow configuration of a deformable internal retainer 118, as described in FIG. 18 and shown in FIG. 76. In some embodiments, pulling the percutaneous transabdominal port with adequate force may withdraw the deformable internal retainer 118 into the abdominal tract 102. The mechanical force exerted on the outer surface 148 of the internal retainer 118 by the abdominal tract may deform the internal retainer 118 to its narrow configuration. The percutaneous transabdominal port is then removed (FIG. 77).

A method of using the percutaneous transabdominal port is disclosed. An optional closure cap 198 is opened. An instrument 128 such as a catheter is then passed through the channel 114 within the hollow tube 100 (FIG. 62). The instrument 128 may be further passed through an aperture 156 or 168 in the internal retainer 118 and into the organ 108. In embodiments where the percutaneous transabdominal port contains a seal 120, the instrument passes through this seal. The instrument 128 may then be withdrawn from the percutaneous transabdominal port. An optional closure cap 198 may then be closed when the port is not is use.

Several precautions ensure safe and correct insertion and removal of the percutaneous transabdominal port. If the percutaneous transabdominal port does not advance easily during insertion 234 through the abdominal tract 102, one may remove the percutaneous transabdominal port and not attempt to advance against resistance with increasing pressure. After insertion 234 of the narrowed internal retainer 118 into the abdominal tract 102 and conversion 236 to a wide configuration, one may gently advance and withdraw 238 the percutaneous transabdominal port to ensure the internal retainer 118 is deployed within the organ 108, and not within the abdominal tract 102. A correctly placed percutaneous transabdominal port advances easily and withdraws until the internal retainer 118 abuts the interior surface of the organ 108 where it joins the opening of the abdominal tract 102. In the case of a mispositioned percutaneous transabdominal port, the straightening rod 170 may be re-inserted to narrow the internal retainer 118. Alternatively, the percutaneous transabdominal port may be withdrawn from the tract 102. During removal, if the percutaneous transabdominal port does not withdraw easily attempts should not be made to withdraw with increasing force. For embodiments where the closure cap 198 is not integrally formed with the hollow tube 100, such as that described in Example 2, secure attachment of the closure cap 198 and external retainer 186 to the hollow tube 100 is ensured prior to inserting the straightening rod 170, to prevent dislodgement of the hollow tube 100 and internal retainer 118 into the organ 108.

### Combinations

The percutaneous transabdominal port may be made and used with many combinations of the components and features described herein. Among combinations of features anticipated by the inventor are:

A percutaneous transabdominal port designated A adapted for access to visceral hollow organs through a tract through an abdominal wall including a hollow tube through which instrumentation accesses internal organs. The hollow tube has an internal end and an external end, and an outer diameter small enough to fit through a tract through an abdominal wall. The port also includes an internal retainer and an external retainer. The internal retainer is connected with the internal end of the hollow tube and has a first configuration with a first outer diameter small enough to fit through the tract and a second configuration with a second outer diameter greater than the first outer diameter.

A port designated AA includes the port designated A wherein the internal retainer is inflatable and deflatable.

A port designated AB includes the port designated A or AA and further includes an integrated check valve permitting inflation (or other inflation means) attached to the external end of the port.

A port designated AC includes the port designated A, AA, or AB and further includes channel which fluidly connects the inflatable internal retainer with the inflation means.

A port designated AD includes the port designated A, AA, AB, or AC wherein the internal retainer is inflated to assume the second configuration and deflated to assume the first configuration.

A port designated AE includes the port designated A wherein the internal retainer is resiliently deformable.

A port designated AF includes the port designated A or AE wherein the internal retainer assumes the first configuration when a mechanical force is applied to an outer surface of the internal retainer and returns to the second configuration upon removal of the force.

A port designated AG includes the port designated A, AE, or AF wherein a mechanical force is applied by a sheath, trocar, or capsule that deforms the internal retainer from the second configuration to the first configuration.

A port designated AH includes the port designated A or AE wherein the internal retainer assumes the first configuration when a mechanical force is applied to an interior surface of the internal retainer and returns to the second configuration upon removal of the force.

A port designated AJ includes the port designated A, AE, or AH wherein a mechanical force is applied by a straightening rod configured to deform the internal retainer from the second configuration to the first configuration.

A port designated AK includes the port designated A, AA, AB, AC, AD, AE, AF, AG, AH, or AJ and further includes a seal to prevent fluid reflux through the hollow tube.

A port designated AL includes the port designated AK wherein a seal is integrally formed with, or attached to, an internal retainer, and provides elastomeric seal to the instrumentation inserted through the hollow tube.

A port designated AM includes the port designated AK wherein a seal is integrally formed with, or attached to, the hollow tube, and provides elastomeric seal to the instrumentation inserted through the hollow tube.

A port designated AN includes the port designated AK, AL, or AM wherein a seal is integral with, or attached to, the hollow tube and the internal retainer, and provides elastomeric seal to the instrumentation inserted through the hollow tube.

A port designated AO includes the port designated AK, AL, AM, or AN wherein a seal is selected from the group comprising one or more of: a duckbill valve, one or more leaflets, a cross-slit valve, a diaphragm with opening that limits fluid flow, and a one-way flapper valve.

A port designated AP includes the port designated A, AA, AB, AC, AD, AE, AF, AG, AH, AJ, AK, AL, AM, AN, or AO and further includes an external retainer that connects with the hollow tube and prevents the hollow tube from further entry inside the abdominal wall.

A port designated AQ includes the port designated AP wherein the external retainer is integrally formed with the hollow tube.

A port designated AR includes the port designated AP wherein the external retainer is assembled with the hollow tube.

A port designated AS includes the port designated AP, AQ, or AR wherein the external retainer has a collar forming an aperture to accommodate the hollow tube and a flange that abuts against an external surface of the abdominal wall.

A port designated AT includes the port designated AP, AR, or AS wherein the external retainer couples with the hollow tube by one or more of: friction; a one-way ratchet; angled protrusions or angled grooves; pushpin locking mechanism; snap-fit locking mechanism; twist-locking mechanism; and suturing.

A port designated AU includes the port designated A, AA, AB, AC, AD, AE, AF, AG, AH, AJ, AK, AL, AM, AN, AO, AP, AQ, AR, AS, or AT which further includes a closure cap to seal the hollow tube external to the abdominal wall.

A port designated AV includes the port designated AU wherein the closure cap is integrally formed with an external retainer.

A port designated AW includes the port designated AU wherein the closure cap is integrally formed with the hollow tube.

A method designated B of inserting a percutaneous transabdominal port through an abdominal wall into an organ, comprising converting an internal retainer to a narrow first configuration, inserting the internal retainer connected to a hollow tube through a tract of the abdominal wall and into an organ, and converting the internal retainer to a wide second configuration.

A method designated BA including the method designated B further comprising shortening the hollow tube to customize the size of the percutaneous transabdominal port to a particular abdominal wall thickness.

A method designated C of using a percutaneous transabdominal port, comprising inserting instrumentation through the percutaneous transabdominal port into the organ, and withdrawing instrumentation through the percutaneous transabdominal port and out of the body.

A method designated CA including the method designated C further comprising inserting instrumentation through a seal of the percutaneous transabdominal port.

A method designated D of removing a percutaneous transabdominal port, comprising converting an internal retainer to a narrow first configuration and withdrawing the percutaneous transabdominal port out of the organ, through the abdominal tract and out of the body.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween. It is also anticipated that steps of methods may be performed in an order different from that illustrated and still be within the meaning of the claims.

1-9. (canceled)

10. A percutaneous transabdominal port, comprising:
a hollow tube with an internal end and an external end, the hollow tube having an outer diameter small enough to fit through a tract through an abdominal wall;
a resiliently deformable internal retainer formed integral with the internal end of the hollow tube and having a first configuration with a first outer diameter small enough to fit through the tract and a second configuration with a second outer diameter greater than the first outer diameter;
wherein the transabdominal port is configured to permit instrumentation to access internal organs through the hollow tube; and
an elastomeric seal configured to seal around objects inserted through the hollow tube; and
wherein the internal retainer is configured to deform from the second configuration to the first configuration upon application of a mechanical force applied by a straightening rod configured to be inserted through the hollow tube;
and wherein there is an opening at a tip of the internal retainer integral to the hollow tube, the opening aligned with an axis of the hollow tube, and the straightening rod being configured to engage the internal retainer without passing through the opening at the tip of the internal retainer.

11. The port of claim 10, wherein the straightening rod is configured to apply mechanical force against an internal surface of a tip of the internal retainer.

12. The port of claim 11, further comprising a seal to prevent fluid reflux through the hollow tube.

13. The port of claim 12, the seal being integral with, or attached to an internal retainer, and
providing elastomeric seal to the instrumentation inserted through the hollow tube.

14. The port of claim 12, the seal being integral with, or attached to the hollow tube, and
providing elastomeric seal to the instrumentation inserted through the tube.

15. The port of claim 12, the seal being selected from the group comprising one or more of: a duckbill valve, one or more leaflets, a cross-slit valve, a diaphragm with opening that limits fluid flow, and a one-way flapper valve.

16. The port of claim 10, further comprising an external retainer that connects with the hollow tube and prevents the hollow tube from further entry inside the abdominal wall.

17. The port of claim 16, wherein the external retainer is integrally formed with the hollow tube.

18. The port of claim 16, the external retainer having a collar forming an aperture to accommodate the hollow tube and a flange that abuts against an external surface of the abdominal wall.

19. The port of claim 18, wherein the external retainer couples with the hollow tube by one or more of: friction; a one-way ratchet; angled protrusions or angled grooves; a pushpin locking mechanism; a snap-fit locking mechanism; a twist-locking mechanism; and suturing.

20. The port of claim 10, further comprising a closure cap to seal the hollow tube external to the abdominal wall.

21. The port of claim 20, the closure cap being integrally formed with an external retainer.

22. The port of claim 20, the closure cap being integrally formed with the hollow tube.

23. A method of inserting a percutaneous transabdominal port through an abdominal wall into an organ, comprising:
converting an internal retainer to a narrow first configuration by inserting a straightening rod through a hollow tube of the percutaneous transabdominal port to the internal retainer and applying pressure through the straightening rod to the internal retainer;
inserting the internal retainer, connected to a hollow tube having an elastomeric seal configured to seal around objects inserted through the hollow tube, through a tract of the abdominal wall and into an organ; and
converting the internal retainer to a wide second configuration by removing the straightening rod;
wherein there is an opening at a tip of the internal retainer, the opening aligned with an axis of the hollow tube, and the straightening rod configured to engage the internal retainer without passing through the opening at the tip of the internal retainer.

24. The method of claim 23, further comprising shortening the hollow tube to customize size of the percutaneous transabdominal port to a particular abdominal wall thickness.

25. The method of claim 23, further comprising:
inserting instrumentation through the percutaneous transabdominal port into the organ; and
withdrawing instrumentation through the percutaneous transabdominal port and out of the body.

26. The method of claim 25, further comprising inserting instrumentation through a seal of the percutaneous transabdominal port.

27. A method of inserting and removing a percutaneous transabdominal port, comprising:
the method of claim 23,
converting an internal retainer to a narrow first configuration by reinserting the straightening rod through a hollow tube of the percutaneous transabdominal port to the internal retainer and applying pressure through the straightening rod to the internal retainer; and
withdrawing the percutaneous transabdominal port out of the organ, through the abdominal tract and out of the body.

28. The percutaneous abdominal port of claim 10 wherein the straightening rod has a larger diameter than a diameter of the axial hole
